(19) United States
(12) Patent Application Publication
(10) Pub. No.: US 2026/0256490 A1
(43) Pub. Date: Sep. 3, 2026

(54) CONTROLLABLY TRANSLATABLE TISSUE CUTTING DEVICES

(71) Applicant: Highridge Medical, LLC, Westminster, CO (US)

(72) Inventor: Eugene Shoshtaev, Del Mar, CA (US)

(21) Appl. No.: 19/653,367

(22) Filed: Apr. 21, 2026

Related U.S. Application Data
(63) Continuation of application No. 17/214,521, filed on Mar. 26, 2021, now abandoned.
(60) Provisional application No. 63/000,070, filed on Mar. 26, 2020.

(51) Int. Cl. A61B 17/32; A61B 17/00

(52) U.S. Cl. CPC A61B 17/32002; A61B 2017/00261; A61B 2017/320028

(57) ABSTRACT

Tissue cutters are provided, along with systems and methods of using the tissue cutters, and a corresponding trial sizer. A channel cutter and a rasp are also provided, each for use with the tissue cutter or alone. A tissue cutter can be a sweeping cutter, an expansion cutter, a pull cutter, or a push cutter. A sweeping cutter, for example, can include a cutting element having a geared body and a cutting flange; the geared body having a central aperture, and the cutting flange having a leading edge, a trailing edge, and a central opening. The tissue cutter can also include a housing for the cutting element.

### CROSS-REFERENCE TO RELATED APPLICATIONS

This application claims priority to and the benefit of US Application No. 17/214,521, filed Mar. 26, 2021, which claims priority to and the benefit of US Provisional Application No. 63/000,070, filed Mar. 26, 2020, both of which are hereby incorporated herein by reference in their entirety.

### FIELD

The present disclosure relates generally to surgical cutting devices, and more specifically to an instrument for cutting disc material from an intervertebral disc space.

### BACKGROUND

Surgeons need cutting devices that can assist them in cutting and removing tissue from areas adjacent to critical tissues that should not be cut. For example, cutting devices often need to be moved to, and removed from, a surgical target area that is adjacent to, or beyond, such sensitive and critical tissues that should not be cut by the cutting devices. An intervertebral disc is a good example of such a surgical target area. The intervertebral disc is a fibrocartilage structure that lies between adjacent vertebrae in the spine, having a inner gel-like center called the nucleus pulposus which is surrounded by an outer fibrous ring called the annulus fibrosus. Intervertebral discs often need to be removed, either in whole or in part, and they are adjacent to other critical tissues including blood vessels and nerves. A discectomy can be done to remove all or part of a disc, and this may be recommended in cases of disc herniation or degeneration, conditions which can result in a bulging disc or other anatomy pressing on a spinal nerve, for example, causing pain, weakness, or loss of feeling in the legs or back. An operative corridor is established through the patient’s skin and musculature and is used to access the target intervertebral disc with a discectomy instrument, such as the cutting devices taught herein. The process of passing the discectomy instrument through the surgical corridor places critical surrounding tissues at risk, and the cutting devices taught herein help protect these tissues, as well as provides additional control to the process of cutting tissue. As such, one of skill in the art will appreciate having improved cutting devices that provide controlled cutting conditions, namely (i) protection of the sensitive surrounding tissues from the cutting element during placement of the cutting device through the surgical corridor, around sensitive tissues; (ii) an accurate and controlled movement and translation of the cutting element in the disc space to cut disc tissue; and, (iii) protection of the sensitive surrounding tissues from the cutter element when removing the cutting device from the subject.

### SUMMARY

Controllably translatable tissue cutting devices are provided herein. The devices include “sweeping” tissue cutters, “expansion” cutters, “pull” cutter, and “push” cutters. Channel cutters, rasp cutters, and trial sizers are also provided. The cutting devices are explained in the context of removing disc tissue by way of example, although the skilled artisan will appreciate that the cutters taught herein can be configured and used in other target sites without departing from the scope of the disclosure.

In some embodiments, a tissue cutter comprises an elongated body having a longitudinal axis, and comprising at least one depth stop configured to abut at least one vertebral body adjacent the intervertebral disc; a spacing element sized and configured to substantially fill an initial discectomy channel formed by the tissue cutter; and, a cutting element comprising one or a plurality of cutting flanges, each cutting flange having at least one cutting edge for expanding the initial discectomy channel.

In some embodiments, the cutting element can be positioned at least partially within an internal cavity of the spacing element.

In some embodiments, the cutting element comprises at least one cutting flange positioned along a lateral edge of the elongated body and transverse to the longitudinal axis, the at least one cutting flange comprising a distally oriented leading cutting edge and a proximally oriented trailing edge.

In some embodiments, the cutting element further comprises a geared body having a central axis; the spacing element further comprises a rotational connection with the cutting element; and, the tissue cutter further comprises a first cannula in operable connection with the internal cavity and a drive element positioned within the first cannula and in operable contact with the geared body of the cutting element, the drive element configured for pivoting the geared body in the internal cavity for a sweeping arc motion of the cutting flange when actuating the drive element.

In some embodiments, the tissue cutter comprises a second cutting element positioned at least partially within the internal cavity, the second cutting element having a geared body in operable connection with the drive element and a cutting flange comprising a cutting edge. In some embodiments, the cutting element and the second cutting element are positioned on a same side of the spacing element. And, in some embodiments, the cutting element and the second cutting element are positioned on opposing sides of the spacing element. In some embodiments, the drive element is configured to pivot the cutting element and the second cutting element simultaneously.

In some embodiments, the tissue cutter comprises an elongated body having a longitudinal axis; and, a cutting element comprising one of: (a) one or a plurality of cutting flanges, each cutting flange comprising at least one cutting edge, wherein the at least one cutting edge is selected from the group consisting of a distally oriented cutting edge that is transverse to the longitudinal axis, a proximally oriented cutting edge that is transverse to the longitudinal axis, and a combination thereof; or (b) a lateral cutting edge that is oriented parallel to the longitudinal axis.

In some embodiments, the tissue cutter further comprising a spacer having an elongated body and a depth stop, and wherein the cutting element extends away from the depth stop and comprises a cutting flange comprising a distally oriented cutting edge that is transverse to the longitudinal axis, or a proximally oriented cutting edge that is transverse to the longitudinal axis, and wherein, the cutting edge is configured to cut tissue when the tissue cutter is translated along a cutting path from one of proximal to distal or distal to proximal along the elongated body such that the at least one cutting flange contacts with the intervertebral disc to cut the disc tissue along the cutting path.

In some embodiments, the tissue cutter further comprises an expandable cutting head having separable cutting elements operably attached to a wedge assembly having a proximal wedge and a distal wedge; and, an inner shaft operably attached to the distal wedge and translatable within an outer shaft operably attached to the proximal wedge; wherein, the wedge assembly laterally expands the separable cutting elements when the proximal wedge and distal wedge are translated relative to one another through a translation of the inner shaft with respect to the outer shaft, and wherein the cutting elements comprise a lateral cutting edge that is oriented parallel to the longitudinal axis.

In some embodiments, a method of removing a disc tissue from an intervertebral disc of a subject is provided. The method can comprise the steps of creating an initial discectomy channel having a first width; and, inserting a tissue cutter at least partially into the initial discectomy channel. In some methods, the tissue cutter can comprise an elongated body having a longitudinal axis and comprising at least one depth stop configured to abut at least one vertebral body adjacent the intervertebral disc; a spacing element sized and configured to substantially fill the initial discectomy channel; and, a cutting element comprising one or a plurality of cutting flanges, each cutting flange comprising at least one cutting edge. In some embodiments, the methods can include moving the cutting flange into contact with and through the intervertebral disc to cut contacted disc tissue; removing the tissue cutter from the discectomy channel; and, removing the remaining cut disc tissue from the subject to thereby expand the discectomy channel from the first width to a second width.

In some embodiments, the cutting element can be positioned at least partially within an internal cavity of the spacing element. And, in some embodiments, the cutting element is oriented transverse to the longitudinal axis and comprises a cutting flange comprising a leading cutting edge, and a trailing edge; the cutting element further comprises a geared body having a central axis; the spacing element further comprises a rotational connection with the cutting element; and, the tissue cutter further comprises a first cannula in operable connection with the internal cavity and a drive element positioned within the first cannula and in operable contact with the geared body of the cutting element, the drive element configured for pivoting the geared body in the internal cavity for a sweeping arc motion of the cutting flange when actuating the drive element.

In some embodiments, the step of moving the cutting flange into contact with and through the intervertebral disc to cut contacted disc tissue comprises actuating the drive element to pivot the geared body so that the cutting flange travels through the intervertebral disc in a sweeping arc motion.

In some embodiments, the tissue cutter comprises a second cutting element positioned at least partially within the internal cavity, the second cutting element having a geared body in operable connection with the drive element and a cutting flange comprising a cutting edge. In some embodiments, the cutting element and the second cutting element are positioned on a same side of the spacing element. In some embodiments, the cutting element and the second cutting element are positioned on opposing sides of the spacing element. And, in some embodiments, the drive element is configured to pivot the cutting element and the second cutting element simultaneously.

In some embodiments, the step of moving the cutting flange into contact with and through the intervertebral disc to cut contacted disc tissue comprises moving the cutting flange in a sweeping arc motion through the intervertebral disc. And, in some embodiments, the cutting element is one of a distally oriented cutting edge that is transverse to the longitudinal axis or a proximally oriented cutting edge that is transverse to the longitudinal axis and is slidably associated with the elongated body.

In some embodiments, the step of moving the cutting flange into contact with and through the intervertebral disc to cut contacted disc tissue comprises translating the cutting element distally along the elongated body such that the cutting flange translates distally through the intervertebral disc parallel to the longitudinal axis.

In some embodiments, the step of moving the cutting flange through the intervertebral disc comprises translating the cutting element proximally along the elongated body such that the cutting flange translates proximally through the intervertebral disc parallel to the longitudinal axis.

### BRIEF DESCRIPTION OF THE DRAWINGS

Many advantages will be apparent to those skilled in the art with a reading of this specification in conjunction with the attached drawings, wherein like reference numerals are applied to like elements.

FIGS. 1A and 1B are perspective views of sweeping tissue cutters, according to some embodiments.

FIG. 2 is another perspective view of the sweeping tissue cutter of FIG. 1A, according to some embodiments.

FIGS. 3A and 3B are cross-sectional views of a distal end of the sweeping tissue cutters of FIGS. 1A and 1B, according to some embodiments.

FIG. 4 is an exploded view of the sweeping tissue cutter of FIG. 1A, according to some embodiments.

FIG. 5 is a perspective view of an inner shaft member forming part of the sweeping tissue cutter of FIG. 1A, according to some embodiments.

FIG. 6 is a perspective view of a first cannula forming part of the of the sweeping tissue cutter of FIG. 1A, according to some embodiments.

FIGS. 7-8 are perspective views of a second cannula forming part of the sweeping tissue cutter of FIG. 1A, according to some embodiments.

FIGS. 9-10 are perspective views of a cutting element forming part of the sweeping tissue cutter of FIG. 1A, according to some embodiments.

FIGS. 11-12 are perspective views of a first control member forming part of the sweeping tissue cutter of FIG. 1A, according to some embodiments.

FIGS. 13-14 are perspective views of a second control member forming part of the sweeping tissue cutter of FIG. 1A, according to some embodiments.

FIGS. 15-16 are a perspective views of a housing element forming part of the sweeping tissue cutter of FIG. 1A, according to some embodiments.

FIG. 17 is a side plan view of the sweeping tissue cutter of FIG. 1A at one stage of use, according to some embodiments.

FIGS. 18-19 are cross-sectional top plan views of the sweeping tissue cutter of FIG. 1A at other stages of use, according to some embodiments.

FIG. 20 is a side plan view of the sweeping tissue cutter of FIG. 1A at another stage of use, according to some embodiments.

FIGS. 21-22 are cross-sectional top plan views of the surgical cutting device of FIG. 1 at other stages of use, according to some embodiments.

FIG. 23 is an exploded view of another example of a bilateral sweeping tissue cutter, according to some embodiments.

FIG. 24 is a perspective view of the bilateral sweeping tissue cutter of FIG. 23, according to some embodiments.

FIG. 25 is a perspective view of an inner shaft member forming part of the bilateral sweeping tissue cutter of FIG. 23, according to some embodiments.

FIG. 26 is a perspective view of a housing member forming part of the bilateral sweeping tissue cutter of FIG. 23, according to some embodiments.

FIGS. 27-29 are top cross-sectional views of the bilateral sweeping tissue cutter of FIG. 23 at various stages of use, according to some embodiments.

FIG. 30 is a perspective view of the bilateral sweeping tissue cutter of FIG. 23 in conjunction with an alternative example of a housing member, according to some embodiments.

FIG. 31 is a perspective view of the distal end of another embodiment of a second cannula forming part of the sweeping tissue cutters taught herein, according to some embodiments.

FIG. 32 is a perspective view of the bilateral sweeping tissue cutter of FIG. 23 in conjunction with an alternative example of a second cannula, according to some embodiments.

FIG. 33 is a perspective view of the distal end of another example of a second cannula forming part of the sweeping tissue cutters taught herein, according to some embodiments.

FIG. 34 is a side plan view of FIG. 33, according to some embodiments.

FIG. 35 is a side plan view of an alternative example of a second cannula forming part of the sweeping tissue cutters taught herein, according to some embodiments.

FIG. 36 is an exploded perspective view of the second cannula of FIG. 35, according to some embodiments.

FIGS. 37-40 are perspective views of alternative examples of cutting elements forming part of the sweeping tissue cutters taught herein, according to some embodiments.

FIG. 41 is a perspective view of an example of a trial sizer configured for use with the sweeping tissue cutters taught herein, according to some embodiments.

FIG. 42 is a perspective view of an example of a rasp element configured for use with the sweeping tissue cutters taught herein, according to some embodiments.

FIG. 43 is a perspective view of a sweeping tissue cutter having an alternative actuation mechanism, according to some embodiments.

FIG. 44 is a top sectional view of FIG. 43, according to some embodiments.

FIG. 45 is a perspective view of an example of the sweeping tissue cutters taught herein and configured with multiple cutting elements on a side of the cutter, according to some embodiments.

FIG. 46 is a perspective view of an example of the sweeping tissue cutters taught herein and configured with multiple cutting elements on both sides of a bilateral cutter, according to some embodiments.

FIG. 47 is a top sectional view of FIG. 46, according to some embodiments.

FIG. 48 is a perspective view of a channel cutter configured for use with sweeping tissue cutters taught herein, according to some embodiments.

FIG. 49 is a side plan view of the channel cutter of FIG. 48, according to some embodiments.

FIG. 50 is a top plan view of the channel cutter of FIG. 50, according to some embodiments.

FIG. 51 is a perspective view of an example of a rasp instrument, according to some embodiments.

FIG. 52 is a top plan view of the rasp instrument of FIG. 51, according to some embodiments.

FIG. 53 is a side plan view of the rasp instrument of FIG. 52, according to some embodiments.

FIG. 54 is a perspective view of an expansion cutter, according to some embodiments.

FIGS. 55-56 are perspective views of the distal end of the expansion cutter of FIG. 54, according to some embodiments.

FIG. 57 is a top plan view of the distal end of the expansion cutter of FIG. 54, according to some embodiments.

FIG. 58 is an end plan view of the distal end of the expansion cutter of FIG. 54, according to some embodiments.

FIG. 59 is another perspective view of the distal end of the expansion cutter of FIG. 54, according to some embodiments.

FIG. 60 is another top plan view of the distal end of the expansion cutter of FIG. 54, according to some embodiments.

FIG. 61 is another end plan view of the distal end of the expansion cutter of FIG. 54.

FIG. 62 is a perspective view of a pull cutter, according to some embodiments.

FIG. 63 is a cross-sectional view of the pull cutter of FIG. 62, according to some embodiments.

FIG. 64 is a perspective view of a spacer forming part of the pull cutter of FIG. 62, according to some embodiments.

FIG. 65 is a perspective view of a cutter forming part of the pull cutter of FIG. 62, according to some embodiments.

FIGS. 66-69 are cross-sectional top plan views of the pull cutter of FIG. 62 at various stages of use, according to some embodiments.

FIG. 70 is a perspective view of an example of a push cutter, according to some embodiments.

FIG. 71 is a perspective view of a cutter forming part of the push cutter of FIG. 70, according to some embodiments.

FIGS. 72-75 are cross-sectional top plan views of the push cutter of FIG. 70 at various stages of use, according to some embodiments.

### DETAILED DESCRIPTION

One of skill will appreciate that the teachings herein provide controllably translatable cutting devices for cutting tissue. The devices are useful, in particular, in the cutting of intervertebral disc tissue. In some embodiments, variations of a “sweeping” tissue cutter are provided. In some embodiments, variations of an “expansion” cutter are provided. In some embodiments, variations of a “pull” cutter are provided. And, in some embodiments, variations of a “push” cutter are provided. Trial sizers are also provided for the cutting elements. In some embodiments, variations of a “channel” cutter are provided to create the initial discectomy channels to open the disc space for introduction of one of the tissue cutters taught herein. In some embodiments, variations of a “rasp” cutter are provided for removal of additional tissue around the disc space. And, although the cutting devices are explained in the context of removing disc tissue, the skilled artisan will appreciate that the cutters taught herein can be configured and used in other target sites of a subject or patient without departing from the scope of the disclosure.

The term “subject” and “patient” can be used interchangeably in some embodiments and refer to any animal such as a mammal including, but not limited to, non-primates such as, for example, a cow, pig, horse, cat, dog; and primates such as, for example, a monkey or a human. As such, the terms “subject” and “patient” can also be applied to non-human biologic applications including, but not limited to, veterinary, companion animals, commercial livestock, and the like. Moreover, terms of degree are used herein to provide relative relationships between the position and/or movements of components of the systems taught herein. For example, the phrase “at least substantially”, for example, “at least substantially parallel” or “at least substantially along an axis” is used to refer to a position of one component relative to another. As an example, an axis that is at least substantially parallel to another axis can refer to an orientation that is intended, for all practical purposes to be parallel, but it is understood that this is just a convenient reference and that there can be variations due to stresses internal to the system and imperfections in the devices and systems. Likewise, the phrase “at least substantially on a…plane” refers to an orientation or movement that is intended, for all practical purposes to be on or near the plane as a convenient measure of the orientation or movement, but it is understood that this is just a convenient reference and that there can be variations due to stresses internal to the system and imperfections in the devices and systems. Likewise, the phrase “at least substantially coincident” can refer to an orientation or movement that is intended, for all practical purposes to be on or near, for example, an axis or a plane as a convenient measure of the orientation or movement, but it is understood that this is just a convenient reference and that there can be variations due to stresses internal to the system and imperfections in the devices and systems. Other phrases used herein include, but are not limited to, “at least substantially maximize the area” and “at least substantially normal to” and have meanings consistent with those provided above in the context of the instant teachings.

The tissue cutters provided herein can include an elongated body component at least substantially axially aligned with a cutter component. The elongated body, for example, can have a long axis and a depth stop configured to engage at least one vertebral body, and a cutter that is translatable along the axis. The elongated body can take most any form. For example, the elongated body can be a shaft in some embodiments. Likewise, the depth stop can take most any form, as long as it is configured to engage with at least one vertebral body to help create the controlled pressure on the cutter to help provide the controlled translation for the cutting. The cutter can have a cutting element positioned transverse to the axis, the cutting element having a cutting edge and a trailing edge.

In some embodiments, the position of the cutting element on the cutter can be described by the position of a plane dissecting the cutting element and separating the leading edge from the trailing edge of the cutting element. The position of the plane can be defined as having a tilt and a rotation, for example, where 0^{o} tilt is where the plane of the cutting element is positioned at 90^{o} from, or at least substantially normal to, the long axis of the long axis of the elongated body. Likewise, 0^{o} rotation is where the plane of the cutting element is positioned to at least substantially maximize the area of the cutting element passing through the tissue. The amount of tilt can be defined in terms of proximal tilt or distal tilt, for example, and the amount of rotation can be defined as clockwise or counterclockwise rotation of the cutting element from the maximum area of passage. In some embodiments, the cutting element forms a plane that can be positioned about 90 +/- 20 degrees to the long axis of the elongated body, which could be described as a 0^{o} tilt +/- 20^{o}. In these embodiments, the rotation could be 0^{o} +/- 20^{o}, for example. In some embodiments, the tilt can be 0^{o}, 2^{o}, 4^{o}, 6^{o}, 8^{o}, 10^{o}, 12^{o}, 14^{o}, 16^{o}, 18^{o}, 20^{o}, or any amount therein in increments of 0.1^{o}, whether proximal or distal. Likewise, in some embodiments, the rotation can be 0^{o}, 2^{o}, 4^{o}, 6^{o}, 8^{o}, 10^{o}, 12^{o}, 14^{o}, 16^{o}, 18^{o}, 20^{o}, or any amount therein in increments of 0.1^{o}, clockwise or counterclockwise. One of skill will appreciate that the amount of tolerance in the positioning of the cutting element for the cutting will depend on the configuration of the blade, and one of skill will appreciate that there are a plethora of configurations that may be used.

An example use for the cutters is the removal of disc tissue, although one of skill will appreciate that there are other uses. The methods of removing a disc tissue from an intervertebral disc of a subject can include, for example, obtaining the tissue cutter; creating an initial discectomy channel having a first width; inserting the tissue cutter into the discectomy channel to the desired depth using the depth stop; translating the cutter with respect to the depth stop to increase the discectomy channel to a second width; removing the tissue cutter from the discectomy channel; and, removing the disc tissue from the subject.

In each of the embodiments provided herein, a kit can be assembled by offering the cutter with convenient tools that assist in using the cutter. Example tools include a channel cutter for opening the initial discectomy channel, and a rasp for removing tissue around adjacent areas, such as vertebral endplates. For example, a tissue cutting system can be assembled to include a sweeping tissue cutter and a channel cutter having a cross-sectional area sized to cut a channel out of a tissue for entry of the sweeping tissue cutter. Each of the systems can be configured to include a housing for the cutting element for at least substantially protecting the surrounding tissues, critical surrounding tissues that are not intended to be cut, from contact with the cutting element as it is introduced into the patient.

### Sweeping tissue cutters

Generally speaking, the sweeping tissue cutters have an elongated body that carries a cutting element to a target location for cutting. In some embodiments, the cutting element can rotate in position from 0^{o} to 180^{o} with respect to the long axis of the elongated body, where a position for cutting is at about 90^{o} in some embodiments. The cutting element can be delivered to the target cutting site, and the cutting element is then rotated into a cutting position, after which a depth stop can be engaged with a vertebral body to apply a pressure that can be used for a controllable translation of the cutting element through the tissue.

As such, in the sweeping cutters, the amount of sweep can range from about 0^{o} to about 180^{o}, in full or in part, depending on the design of the cutter. In some embodiments, the amount of sweep available may be limited by the particular cutting device configuration. For example, the device may stop at a desired cutter element position transverse to the long axis of the cutting device. In some embodiments, the stop position can range from perhaps 70^{o} to 110^{o}, 75^{o} to 105^{o}, 80^{o} to 100^{o}, 85^{o} to 95^{o}, 87^{o} to 93^{o}, or any range therein in increments of 1^{o}. In some embodiments, the cutting device comprises a control mechanism configured to ensure rotational sweep of the cutting element stops at a 90º cutting orientation, or at least substantially 90^{o}, that is, a 90^{o} tilt, placing the cutting element transverse to the long axis of the cutting device. In some embodiments, in fact, the cutting device may also provide a translational stop during the translational cutting process of translating the cutting element with respect to a depth stop, the translational stop allowing the device to release the 90^{o} tilt stop that was designed to make the cut, and allow the cutting element to continue rotational sweep back into the housing from the 90^{o} position to the 180^{o} position for removal of the cutter from the subject.

In some embodiments, these devices can include a cutting element having a geared body and a cutting flange. The geared body can have a central axis, and the cutting flange can have a leading edge and a trailing edge. These embodiments can include a housing for the cutting element, the housing having a rotational connection with the cutting element. In these embodiments, the housing at least substantially protects the surrounding tissues, critical surrounding tissues that are not intended to be cut, from contact with the cutting element as it is introduced into the patient and removed from the patient through the surgical corridor. The devices can include a first cannula in operable connection with the housing; and, there can also be a drive element positioned within the first cannula and in operable contact with the geared body of the cutting element. The drive element can be configured for pivoting the geared body in the housing for a sweeping arc motion of the cutting flange when actuating the drive element. In some embodiments, the sweeping tissue cutter can also include a second cutting element having a geared body and a cutting flange, the housing having a second rotational connection with the second cutting element. In some embodiments, the leading edge of the cutting flange has a cutting surface configured to cut a tissue during the sweeping arc motion. In some embodiments, the trailing edge of the cutting flange has a cutting surface. In some embodiments, the cutting element has a rasp surface. And, in some embodiments, the sweeping tissue cutter includes a first actuator for actuating the drive element.

The cutting device can include first and second control members positioned near the proximal end of an actuator shaft. In some embodiments, actuating the first control member causes the cutting element to rotate from a first position in which the cutting element is contained within the housing element to a second position in which the cutting element is protruding from the housing element. Once the cutting element is in the second position, the second control member may be actuated to maneuver the distal end of the surgical cutting device axially in a proximal direction to drag the exposed cutting element through the intervertebral disc space, thereby cutting or shaving the disc.

In some embodiments, the actuator shaft comprises an axially aligned, elongated, generally cylindrical member having a proximal end configured to engage the first control member, a distal end including an actuation mechanism configured to engage the cutting element, and a central portion extending between the proximal and distal ends. In some embodiments, the actuator shaft can include a drive element to drive the sweeping movement of the cutting element. In some embodiments, the actuation mechanism can comprise a gear rack having a plurality of ridges or elongated gear teeth extending transverse to the longitudinal axis of the shaft, as a component of a rack and pinion type mechanism, for example, in which the actuation mechanism can be configured to engage the gear teeth of the cutting element such that axial translation of the shaft (e.g. in a proximal and/or distal direction) causes rotational pivoting of the cutting element(s). In some embodiments, the actuation mechanism comprises a worm gear. In some embodiments, the actuation mechanism comprises a linkage mechanism. In some embodiments, the distal end of the shaft further comprises a guide element sized and configured to interact with the housing member to stabilize the distal end of the shaft relative to the housing member during translation of the shaft.

In some embodiments, a sweeping tissue cutter can include a cutting element having a geared body and a cutting flange, for example. The geared body can have a central bore, and the cutting flange can have a leading edge, a trailing edge, and a central opening. In some embodiments, a housing can be included for the cutting element, the housing having a pivot pin in a rotational connection with the central aperture of the cutting element. In some embodiments, drive element can be included in operable contact with the geared body. In some embodiments, the drive element can be configured for pivoting the geared body on the pivot pin for a rotational pivoting of the cutting element with the housing for a sweeping arc motion of the cutting flange when actuating the drive element. The elements of such an embodiment can have different configurations. For example, the “central bore” can include a blind hole, or a through-hole. In some embodiments, the central bore is an aperture.

As noted, a depth stop can be included as a feature that engages with a vertebral body to apply a pressure that can be used to controllable translate the cutting element through the tissue. The depth stop can be configured as a component of a second cannula, for example. As such, in some embodiments, the sweeping tissue cutter can further comprise a second cannula, the first cannula positioned within, and translatable with respect to, the second cannula. The second cannula can further comprise a depth stop configured to engage at least one vertebral body. The engagement of the depth stop with the vertebral body allows for a controlled and accurate application of pressure, resulting in a controlled and accurate translational movement of the cutter in the subject’s target tissue area.

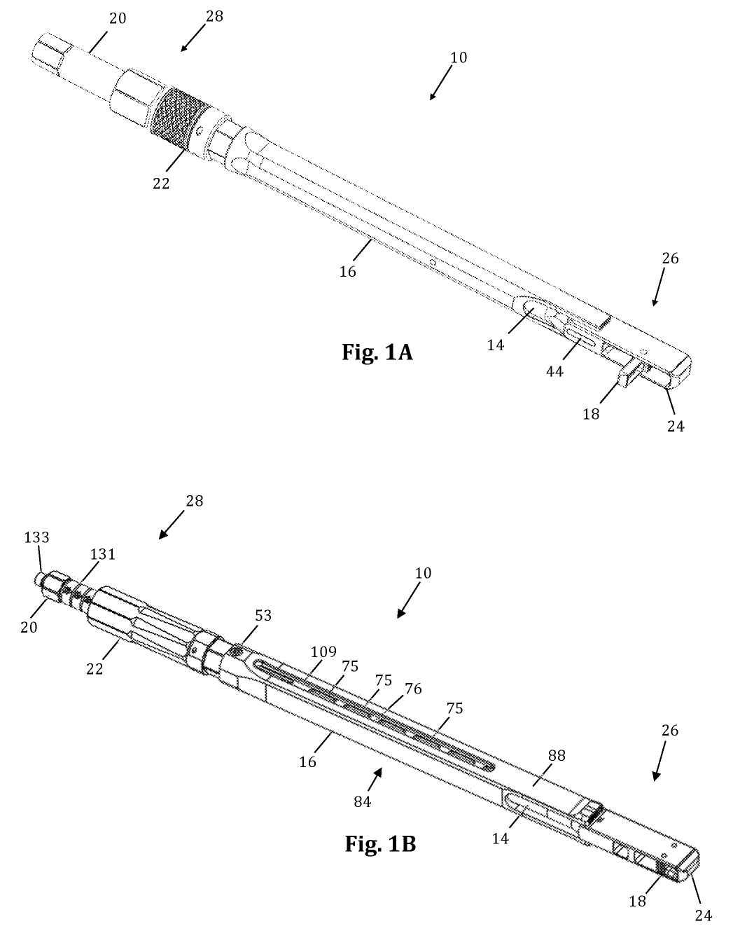

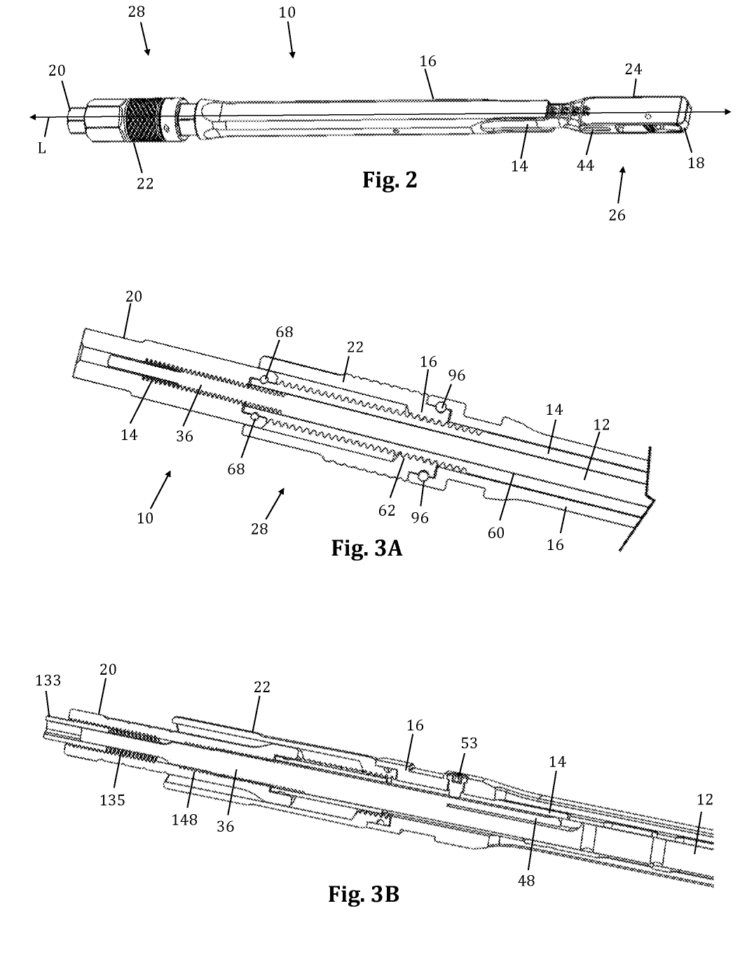

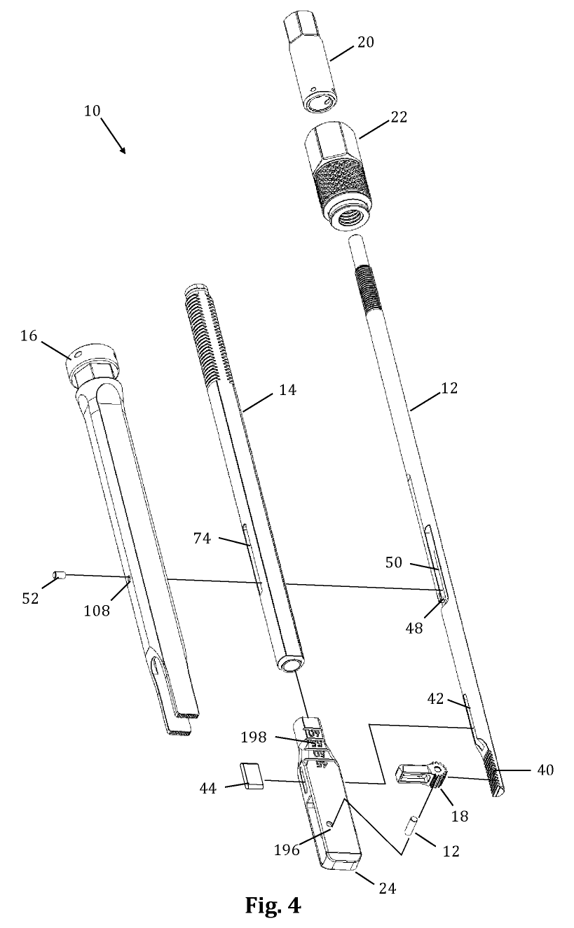

FIGS. 1-4 illustrate an example of a sweeping tissue cutter 10, according to some embodiments. FIGS. 1A and 1B are perspective views of sweeping tissue cutters, according to some embodiments. FIG. 2 is another perspective view of the sweeping tissue cutter of FIG. 1A, according to some embodiments. FIGS. 3A and 3B are cross-sectional views of a distal end of the sweeping tissue cutters of FIGS. 1A and 1B, according to some embodiments. FIG. 4 is an exploded view of the sweeping tissue cutter of FIG. 1A, according to some embodiments. As shown in at least FIGS. 1A, 1B, and 2, for example, the sweeping tissue cutter 10 can be generally axially aligned (e.g. along longitudinal axis L 1) and is sized and configured to extend into a patient to a surgical target site. By way of example, the sweeping tissue cutter 10 can be a unilateral cutting device having the ability to cut tissue material on only one side of the device. As shown in at least FIGS. 3 and 4, the sweeping tissue cutter 10 can include a shaft 12 positioned within a first cannula 14, which is itself positioned within a second cannula 16. As shown in at least FIGS. 1A, 1B, 2, and 4, the sweeping tissue cutter 10 can further include a cutting element 18 positioned near and interacting with the distal end of the shaft 12, and first and second control members 20, 22 positioned near the proximal end of the shaft 12. The cutting element 18 includes the geared body and the cutting flange as shown in FIGS. 1A and 4. As shown in at least FIGS. 1A, 1B, 2, and 4, the first cannula 14 can further include a housing element 24 configured to facilitate insertion of the distal end of the device 10 into an intervertebral disc space while simultaneously protecting the distal end of the shaft 12 including the cutting element 18 from contact with patient tissue during insertion. As shown in at least FIGS. 2, 3A, 3B, and 4, the sweeping tissue cutter 10 has a distal end 26 comprising the distal end of the shaft 12, the cutting element 18, and the housing member 24. The sweeping tissue cutter 10 has a proximal end 28 comprising the respective proximal ends of the shaft 12, first cannula 14, and second cannula 16, as well as the first and second control elements 20, 22. As will be explained, actuating the first control member 20 causes the cutting element 18 to rotate from a first position in which the cutting element 18 is contained within the housing element 24 to a second position in which the cutting element 18 is protruding from the housing element 24. Once the cutting element 18 is in the second position, the second control member 22 may be actuated to maneuver the distal end 26 of the sweeping tissue cutter 10 (e.g. including the shaft 12, cutting element 18, and housing member 24) axially in a proximal direction to drag the exposed cutting element 18 through the intervertebral disc space, thereby cutting or shaving the disc.

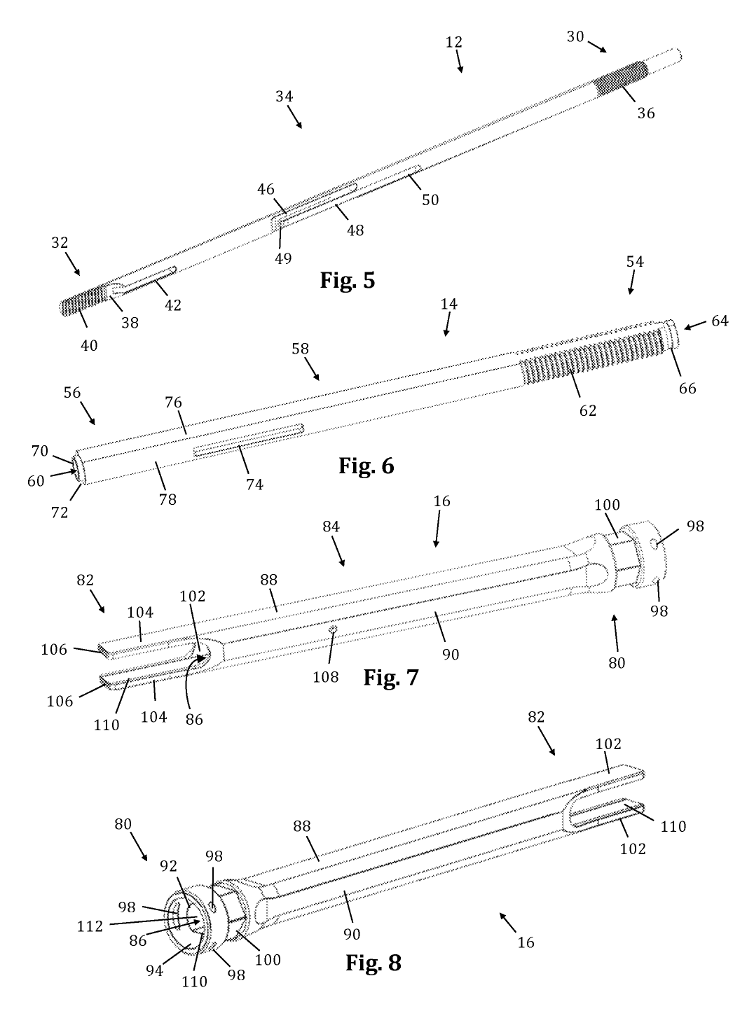

A rotational drive element can be used to rotate the geared body of the cutting element 18. For example, a rack and pinion arrangement can be used in some embodiments. FIG. 5 is a perspective view of an inner shaft member forming part of the sweeping tissue cutter of FIG. 1A, according to some embodiments. It is such a rack and pinion arrangement. The rotational drive element is the shaft 12 having the rack 40 for the pinion gear of cutting element 18. FIG. 5 illustrates an example of the shaft 12 and, by way of example only, the shaft 12 comprises an axially aligned (e.g. along longitudinal axis L 1), elongated, generally cylindrical member having a proximal end 30, distal end 32, and a central portion 34 extending between the proximal and distal ends. The proximal end 30 includes a threaded portion 36 sized and configured to engage the threaded portion 148 of the central lumen 136 of the first control member 20. The distal end 32 includes a cutaway region 38 including a gear rack 40 comprising a plurality of ridges or elongated gear teeth extending transverse to the longitudinal axis L 1. The gear rack 40 is configured to engage the gear teeth 118 of the cutting element 18 such that axial translation of the shaft 12 (e.g. in a proximal and/or distal direction) causes rotational pivoting of the cutting element 18. The distal end 32 of the shaft further comprises an axial recess 42 formed within the shaft, the axial recess 42 configured to receive a portion of a guide member 44 therein (see e.g. FIGS. 1-2 and 4). The guide member 44 is sized and configured to securely mate with the guide aperture 194 of the housing member 24 and is slidingly engaged with the shaft 12 (by way of axial recess 42) to stabilize the distal end 32 of the shaft 12 relative to the housing member 24 during translation of the shaft 12.

The central portion 34 includes a central recess 46 and an axially-aligned cantilever flange 48 extending distally into the central recess 46. The flange 48 has lip 49 positioned at the distal end of the flange 48. At least a portion of the flange 48 including the lip 49 is configured to slidingly couple with the elongate aperture 74 of the first cannula 14. The central portion 34 further includes an axially-aligned elongated recess 50 formed on the outer-facing surface of the flange 48 and extending proximally along the surface of the shaft 12. The elongated recess 50 is sized and configured to slidingly receive at least a portion of a control pin 52. The control pin 52 is sized and configured to securely mate with the lateral aperture 108 of the second cannula 16 and is slidingly engaged with the shaft 12 (by way of elongated recess 50) and the first cannula 14 (by way of elongated aperture 74) to act as link between all three components (e.g. shaft 12, first cannula 14, and second cannula 16) during use (See e.g. FIGS. 1-2 and 4). As will be explained below, the cantilever flange 48 interacts with the elongated aperture 74 and the control pin 52 to control the rotational movement of the cutting element 18. Referring back to FIGS. 1B and 3B, in some embodiments, the sweeping tissue cutter 10 can include a setscrew 53 that interacts with the cantilever flange 48 to control the rotational movement of the cutting element 18. The setscrew 53 can also improve the ease of disassembling the sweeping tissue cutter 10 for cleaning, for example.

FIG. 6 is a perspective view of a first cannula forming part of the of the sweeping tissue cutter of FIG. 1A, according to some embodiments. FIG. 6 illustrates an example of the first or inner cannula 14 and, by way of example only, the first cannula comprises an axially aligned (e.g. along longitudinal axis L 1), elongated, generally tubular member having a proximal end 54, distal end 56, a central portion 58 extending between the proximal and distal ends, an inner lumen 60 extending axially through the entirety of the first cannula 14, a pair of opposing generally planar sides 76, and a pair of opposing generally curved sides 78. The proximal end 54 includes a threaded portion 62 sized and configured to engage the threaded portion 168 of the central lumen 152 of the second control member 22. Due to this threaded engagement, rotation of the second control 22 member causes axial translation (e.g. in a proximal direction) of the first cannula 14 and housing element 24. As such, in some embodiments, the threaded portion 62 can be referred to as a translational drive element on the first cannula, the translational drive element configured for an axial translation of the first cannula within the second cannula, the axial translation of the first cannula causing an axial translation of the tissue cutter when actuating the translational drive element.

The proximal end 54 further includes a proximal aperture 64 providing access to the inner lumen 60 and a circumferential recess 66 positioned between the threaded portion 62 and the proximal aperture 64. The circumferential recess 66 is sized and configured to interact with at least a portion of a retaining element 68 (e.g. ring, coil, pin(s), see FIG. 3) configured to securely associate the first cannula 14 and the first control member 20 while allowing rotational movement of the first control member 20 relative to the first cannula 14. This interaction allows the inner control member 20 to advance the shaft 12 (thereby deploying the cutting element 18) without advancing the first cannula 14 and helps ensure that the shaft 14 (and cutting element 18) moves in concert with the inner cannula 14 (and housing element 24) when the second control member 22 is rotated.

The distal end 56 includes a distal aperture 70 providing access to the inner lumen 60 and a distal lip 72 configured to facilitate engagement between the first cannula 14 and the housing element 24. The central portion 58 includes an elongated aperture 74 formed in one side of the cannula 14, oriented parallel to the longitudinal axis L 1, and opening through to the inner lumen 60. The elongated aperture 74 is configured to allow at least a portion of the control pin 52 to extend therethrough (e.g. generally transverse to longitudinal axis L 1) while providing a guide track to guide translation of the guide pin 52. As will be explained further below, the distal end of the elongated aperture 74 provides a translation stop for the cantilever flange 48 and the control pin 52, which in turn in part controls the rotation movement of the cutting element 18. By way of example only, the inner lumen 60 has a generally cylindrical cross-sectional shape and is sized and configured to receive the shaft 12 therethrough. Referring back to FIG. 1A, in some embodiments, the central portion 58 may include one or more additional (or alternative) elongated apertures 75 formed in at least one of the generally planar sides 76. By way of example only, the elongated apertures 75 may be provided to make the sweeping tissue cutter 10 easier to clean after use.

FIGS. 7-8 are perspective views of a second cannula forming part of the sweeping tissue cutter of FIG. 1A, according to some embodiments. FIGS. 7-8 illustrate an example of a second or outer cannula 16. By way of example only, the second cannula 16 comprises an axially aligned (e.g. along longitudinal axis L 1), elongated, generally tubular member having a proximal end 80, distal end 82, a central portion 84 extending between the proximal and distal ends, an inner lumen 86 extending axially through the entirety of the second cannula 16, a first pair of opposing generally planar sides 88, and a second pair of opposing generally planar sides 90. The proximal end 80 includes a proximal aperture 92 providing access to the inner lumen 86 and a proximal cavity 94 surrounding the proximal aperture 92. The proximal cavity 94 is sized and configured to receive the distal extension 164 of the second control member 22 therein. The distal extension 164 is securely mated with the proximal cavity 94 by way of a retaining element 96 (e.g a plurality of retaining pins, coil, ring, and the like, see FIG. 3) that interacts with a circumferential recess 166 formed in the distal extension 164 and a plurality of apertures 96 (or corresponding circumferential recess, for example) formed in the proximal end 80. While securely mated to the second cannula 16, the second control member 22 is rotationally moveable relative to the second cannula 16. The proximal end 80 further includes an attachment element 100 configured to provide a place of attachment for additional instrumentation, including but not limited to (and by way of example only, a handle member, articulating arm, or robotic arm).

The distal end 82 includes a distal aperture 102 providing access to the inner lumen 86 and a pair of distal extensions 104 that extend the first planar sides 88. In use, the surgical cutter 10 is advanced to a surgical target site and positioned such that the distal extensions 104 contact bone tissue adjacent the target site (e.g. first and second vertebral bodies adjacent the intervertebral disc space targeted for disc removal in the instant example). Thus, the distal ends of the distal extensions 104 include vertebral contact surfaces 106 that may have one or more anti-migration features such as by way of example only grooves, ridges, roughening, pins, spikes, screws, etc. configured to prevent movement of the distal extensions 104 relative to the vertebral bodies when the second cannula 16 is placed in contact therewith.

The central portion 84 includes a lateral aperture 108 configured to securely receive the control pin 52 described above. The inner lumen includes a pair of opposing elongated planar surfaces 110 configured to slidingly engage the opposing planar sides 76 of the first cannula 14 and a pair of opposing elongated curved surfaces 112 configured to slidingly engage the opposing curved sides 78 of the first cannula 14. This shaped engagement prevents rotational movement of the first and second cannulas 14, 16 relative to each other during use. Referring back to FIG. 1A, in some embodiments, the central portion 84 may include an elongated aperture 109 formed in at least one of the first planar sides 88. By way of example only, the elongated aperture 109 may be provided to make the sweeping tissue cutter 10 easier to clean after use.

The sweeping tissue cutter 10 is configured for advancement through an established operative corridor in a patient to a surgical target site. By way of example only, the surgical target site in the instant disclosure is described as an intervertebral disc space, however the sweeping tissue cutter 10 may be used in other target sites without departing from the scope of the disclosure.

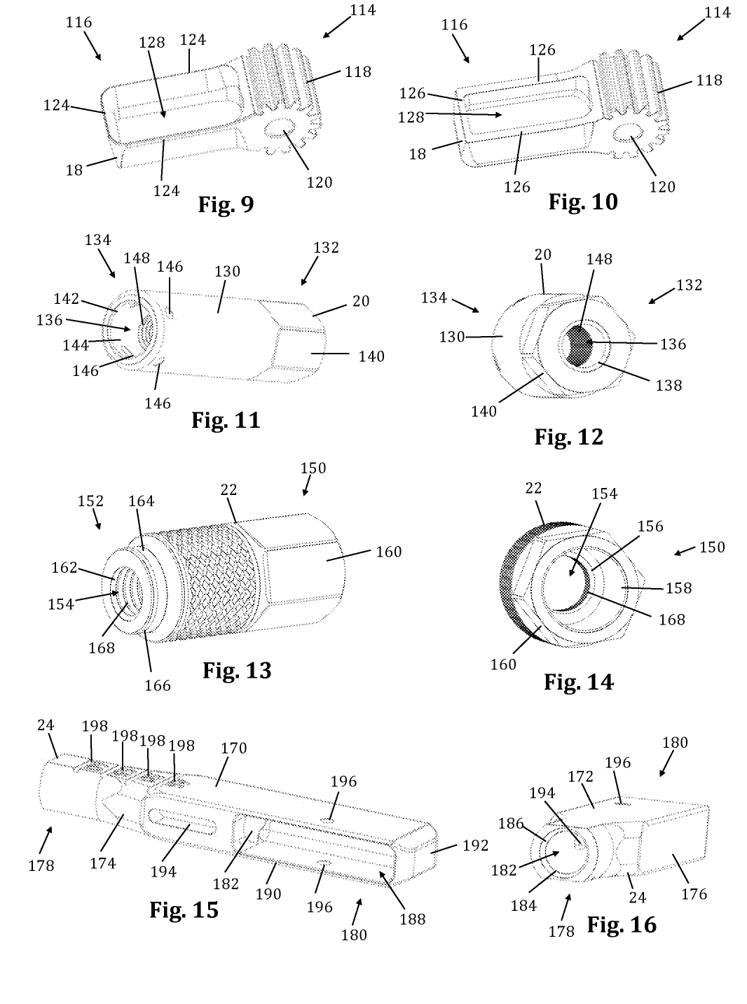

FIGS. 9-10 are perspective views of a cutting element forming part of the sweeping tissue cutter of FIG. 1A, according to some embodiments. FIGS. 9-10 illustrate an example of the cutting element 18. By way of example, the cutting element 18 of the current example embodiment includes a geared body 114 and a cutting flange 116. The geared body 114 is generally cylindrical in shape and comprises a plurality of elongated gear teeth 118 configured to interact with the gear rack 40 of the shaft 12 such that axial translation of the shaft 12 (e.g. in a proximal and/or distal direction) causes rotational pivoting of the cutting element 18, as described above. As such, in the present example the gear teeth 118 are oriented transverse to the longitudinal axis L 1 of the sweeping tissue cutter 10. The geared body 114 further comprises a central aperture 120 configured to receive a pivot pin 122 therethrough (e.g. FIG. 4). The pivot pin 122 is securely mated to the pivot apertures 196 of the housing element 24 (described below) and establishes an axis of rotation about which the cutting element 18 pivots upon deployment. The cutting flange 116 of the current example embodiment comprises a generally rectangular frame having a leading edge 124, trailing edge 126, and a central opening 128. The leading edge 124 comprises a sharp edge configured for cutting through soft tissue (e.g. intervertebral disc material in the current example). The trailing edge 126 comprises a generally planar or dull edge having a width that gives the cutting element a base for stability. The central opening 128 is substantially large (e.g. such that the leading edge 124 represents the perimeter of the opening 128) to allow the cutting element 18 to dislodge a substantial amount of tissue material as the cutting element 18 advances through the tissue without simultaneously removing the tissue from the space.

FIGS. 11-12 are perspective views of a first control member forming part of the sweeping tissue cutter of FIG. 1A, according to some embodiments. FIGS. 11-12 illustrate an example of the first control member 20. By way of example, the first control member 20 comprises a generally cylindrical body, a substantial portion of which has a smooth outer surface 130 sized and configured to nest within the proximal cavity 158 of the second control member 22 (see below). The first control member includes a proximal end 132, distal end 134, and central lumen 136 extending longitudinally (e.g. along longitudinal axis L 1) therethrough. By way of example, the proximal end 132 includes a proximal aperture 138 providing access to the central lumen 136 and a contoured outer surface 140 configured to enable manual manipulation of the first control member 20 during use. The distal end 134 includes a distal aperture 142 providing access to the central lumen 136 and a distal cavity 144 surrounding the distal aperture 142. The distal cavity 144 is sized and configured to receive the proximal end 54 of the first cannula 14 including the circumferential recess 66 and retaining element 68 therein (e.g. FIGS. 3 & 6). The retaining element 68 may be secured to the first control member 14 by any suitable engagement element 146, including but not limited the plurality of retaining apertures (shown by way of example only), or a corresponding circumferential recess, for example. This interaction allows the inner control member 20 to advance the shaft 12 (thereby deploying the cutting element 18) without advancing the first cannula 14 and also ensures that the shaft 14 (and cutting element 18) moves in concert with the inner cannula 14 (and housing element 24) when the second control member 22 is rotated. The central lumen 136 includes a threaded portion 148 configured to threadedly engage the threaded portion 36 of the shaft 12. This threaded engagement drives distal advancement of the shaft 12 upon rotation or actuation of the first control member 20 to deploy the cutting element 18 as described herein.

In some embodiments, depth indicators can be placed more proximal on the cutter devices taught herein for ease of viewing during use. Referring back to FIG. 1, for example, the first control member 20 can further comprise a plurality of depth markings 131 machined into (for example) the smooth outer surface 130. By way of example only, the depth markings 131 can be configured to correspond to depth markings 198 on the housing element 24, described herein with respect to at least FIG. 15. Positioning a set of depth markings 131 on the first control member 20, for example, can be done to make the depth indicators more visible to the user.

In some embodiments, the sweeping tissue cutter 10 further includes a proximal indicator 133 threadedly associated with the central lumen 136 of the first control member 20 to provide the user with a visual indication of the position of the cutting element 18. More specifically, the proximal indicator 133 is associated with a second threaded portion 135 positioned within the central lumen 136 proximal to threaded region 148 and having an opposite threadform to that of the threaded region 148 such that, upon rotation of the first control member 20 in a clockwise direction (for example), the shaft 12 translates distally to deploy the cutting element 18 as described above and the proximal indicator 133 translates proximally to indicate positioning of the cutting element 18. For example, when the cutting element 18 is in the initial position, the proximal indicator 133 may be fully within the central lumen 136 (e.g. in a sub-flush position relative to the proximal end 132). When the cutting element 18 is deployed at the optimal cutting angle (for example 90º), the proximal indicator 133 may be flush with the proximal end 132 of the first control member 20. When the cutting element 18 has rotated fully through the disc, the proximal indicator 133 is fully extended through the proximal aperture 138, as shown by way of example only in FIGS. 1A and 3A.

FIGS. 13-14 are perspective views of a second control member forming part of the sweeping tissue cutter of FIG. 1A, according to some embodiments. FIGS. 13-14 illustrate an example of the second control member 22. By way of example, the second control member 22 comprises a generally cylindrical body having a proximal end 150, distal end 152, and central lumen 154 extending longitudinally (e.g. along longitudinal axis L 1) therethrough. By way of example, the proximal end 150 includes a proximal aperture 156 providing access to the central lumen 154 and a smooth, unthreaded generally cylindrical proximal cavity 158 sized and configured to nestingly receive the first control member 20 therein. The proximal end 150 further comprises a contoured outer surface 160 configured to enable manual manipulation of the second control member 22 during use. The distal end 152 includes a distal aperture 162 providing access to the central lumen 154 and a distal extension 164 surrounding the distal aperture 162. The distal extension 164 is sized and configured to nestingly mate within the proximal cavity 94 of the second cannula 16 such that the circumferential recess 166 receives the retaining element 96 therein (e.g. FIG. 3). The central lumen 154 includes a threaded portion 168 configured to threadedly engage the threaded portion 62 of the first cannula 14. This threaded engagement drives proximal retreat of the first cannula 14 upon rotation or actuation of the second control member 22 to “pull” the cutting element 18 proximally through the disc space (or other surgical site) as described herein. While securely mated to the second cannula 16, the second control member 22 is rotationally moveable relative to the second cannula 16, ensuring that the second cannula 16 remains positioned against the bony structure during use.

FIGS. 15-16 are a perspective views of a housing element forming part of the sweeping tissue cutter of FIG. 1A, according to some embodiments. FIGS. 15-16 illustrate an example of the housing element 24. By way of example only, the housing element 24 comprises opposing first and second sides 170,172, each comprising a generally planar surface, opposing third and fourth sides 174,176, a proximal portion 178, and a distal portion 180. The proximal portion 178 includes an inner lumen 182 extending axially therethrough and configured to be continuous with the inner lumen 60 of the first cannula 14. The proximal portion 178 further comprises a proximal aperture 184 providing access to the inner lumen 182 and a proximal cavity 186 sized and configured to nestingly receive the distal lip 72 therein to seamlessly connect the housing element 24 with the inner cannula 14. The distal portion 180 comprises a chamber 188 wherein the first, second, and third sides 170,172,174 form three sides of the chamber 188. The chamber 188 has a large aperture 190 formed in the fourth side 176 provides access into the chamber 188 from the outside. The chamber is sized and configured to contain and protect the distal end 32 of the shaft 12 and the cutting element 18 when the cutting element 18 is in an initial undeployed position (e.g. 0°, see FIG. 18) as well as when the cutting element 18 is in a final position (e.g. 180°, see FIG. 22). The distal portion 180 further includes a tapered leading end 192 to facilitate advancement of the sweeping tissue cutter 10 through patient tissue. The third side 174 includes a guide aperture 194 sized and shaped to receive the guide member 44 therein. The first and second sides 170,172 each include a pivot aperture 196 configured to secure one end of the pivot pin 122 therein to secure the geared body 114 of the cutting element 12 within the chamber 188.

In some embodiments, the housing element comprises a plurality of depth markings configured to enable presetting of the surgical cutting device at the maximum distal advancement of the housing member based on disc space sized determined by prior use of a trial sizer. The first side 170 can further comprise a plurality of depth markings 198 machined into (for example) the proximal portion 178. Prior to using the sweeping tissue cutter 10 to clean out a disc space (or other target site), a trial sizer may be used to determine the size of the target disc space. The sweeping tissue cutter 10 may then be preset to the marking corresponding to the determined size. For example, if the trial sizer indicated that the size of the disc space is 50mm, then prior to insertion into a patient, the second control member 22 may be rotated to adjust the outer cannula 16 relative to the inner cannula 14 so that the vertebral contact surfaces 106 on the distal extensions 104 are lined up with the depth marking 198 corresponding to 50mm. This presets the sweeping tissue cutter 10 at the maximum distal advancement of the housing member 24, and also determines the distance in which the housing member will translate proximally during use.

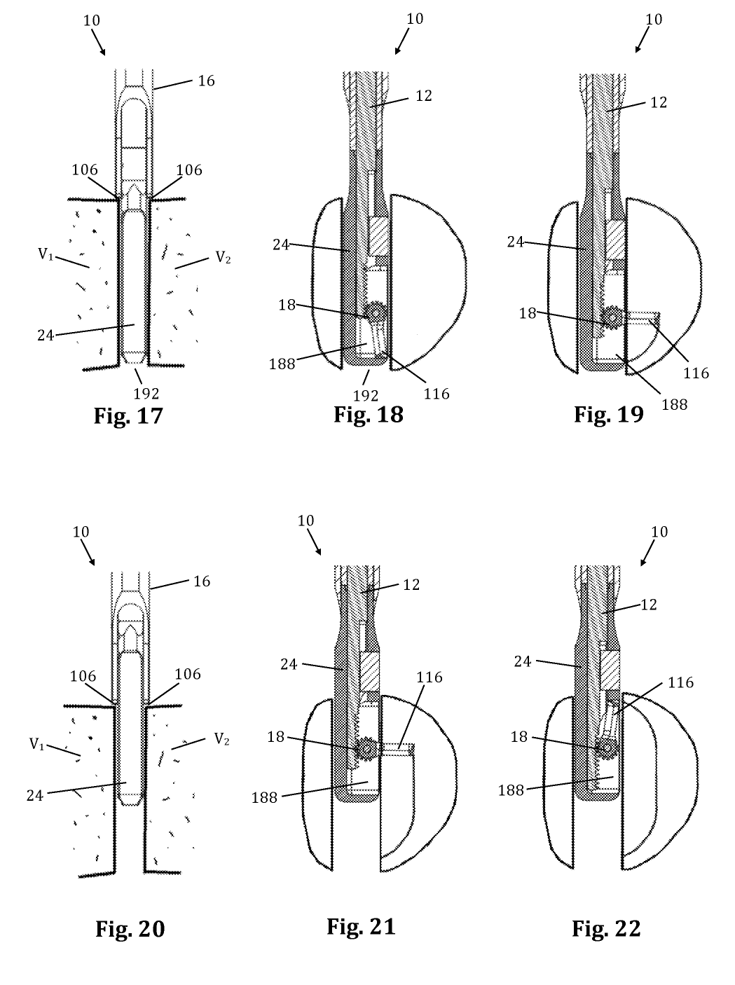

FIGS. 17-22 illustrate the positioning of several components of the sweeping tissue cutter 10 during various stages of a tissue cutting procedure (e.g. discectomy), according to some embodiments. FIG. 17 is a side plan view of the sweeping tissue cutter of FIG. 1A at one stage of use, according to some embodiments. FIGS. 18-19 are cross-sectional top plan views of the sweeping tissue cutter of FIG. 1A at other stages of use, according to some embodiments. FIG. 20 is a side plan view of the sweeping tissue cutter of FIG. 1A at another stage of use, according to some embodiments. FIGS. 21-22 are cross-sectional top plan views of the surgical cutting device of FIG. 1 at other stages of use, according to some embodiments.

By way of example, FIGS. 17 and 20 illustrate a “side” view showing adjacent vertebral bodies V₁, V₂ and the relative positioning of the housing member 24 within the intervertebral disc space, and FIGS. 18-19 and 21-22 illustrate a cross-sectional “top” view of a disc space looking along the spinal column (e.g. cranial-caudal). In use, once the size of the disc space has been determined and the sweeping tissue cutter 10 has been adjusted to the predetermined depth as explained above, the device 10 is advanced through the operative corridor and into the disc space until the vertebral contact surfaces 106 of the leading end 192 of the housing element 24 abut the vertebral bodies V₁, V₂ that are adjacent the target intervertebral disc space. When this happens, the leading end 192 of the housing member 24 will be positioned in the distal-most part of the disc space (FIGS. 17-18).

The next step is to deploy the cutting element 18 from a first or initial position wherein the cutting flange 116 is at 0° relative to the shaft 12 within the chamber 188 to a second or cutting position wherein the cutting flange 116 is at 90° relative to the shaft 12, in some embodiments. This can be accomplished by rotating the first control member 20 as far as the device 10 will allow. The cantilever flange 48 of the shaft 12 and control pin 52 interact with the elongated aperture 74 of the first cannula 14 to ensure the cutting element 18 is in the proper position during disc cutting. For example, in the initial insertion state (e.g. FIG. 18), the control pin 52 extends through the elongated aperture 74 and mates with the elongated recess 50 at a point proximal to the proximal end of the cantilever flange 48, allowing at least a portion of the flange 48 including the lip 49 to extend into the elongated aperture 74. As the first control member 20 is rotated, the shaft 12 is advanced distally relative to the first cannula 14, causing the cutting element 18 to pivot and rotating the cutting flange 116 outward into the disc space (cutting a curved swath of disc in the process). During this distal translation of the shaft 12, the flange 48 including the lip 49 advances distally within the elongated aperture 74 of the first cannula 14. The elongated aperture 74 is sized and configured such that when the distal end of the flange 48 including the lip 49 abuts the distal end of the elongated aperture 74, the first control member 20 is no longer able to be rotated and the cutting flange 116 is positioned at a 90° angle relative to the shaft 12 (e.g. FIG. 19). Thus, the elongated aperture 74 functions as a stop element to ensure the cutting element 18 is properly positioned in the second, or “cutting”, position for disc cutting. It should be appreciated by the skilled artisan that the stop feature merely assists in positioning the blade for cutting, and it is a convenience. As such, the stop feature is an added configuration that can add significant value, but that may or may not be used in the cutting devices taught herein.

FIGS. 17-19 shows that the sweeping action of the cutting element cuts the tissue. That is, a sweeping tissue cutter may cut tissue by the sweeping action alone, in some embodiments. As discussed, a method of removing a disc tissue from an intervertebral disc of a subject can include obtaining the sweeping tissue cutter; creating an initial discectomy channel having a first width; inserting the tissue cutter into the discectomy channel; actuating the cutting element of the cutting head in the sweeping arc motion to cut the disc tissue and increase the discectomy channel to a second width; removing the tissue cutter from the discectomy channel; and, removing the disc tissue from the subject.

However, as shown in FIGS. 20-22, the sweeping tissue cutter can combine the sweeping action with a controlled translation of the cutter to remove even more tissue in a controlled fashion. For example, a method of removing a disc tissue from an intervertebral disc of a subject can include obtaining the sweeping tissue cutter; creating an initial discectomy channel having a first width; selecting a desired depth of entry of the tissue cutter into the disc tissue, the selecting including translating the second cannula axially on the first cannula to the desired depth; inserting the tissue cutter into the discectomy channel to the desired depth; actuating the cutting element of the cutting head in the sweeping arc motion to cut the disc tissue and increase the discectomy channel to a second width; translating the cutter with respect to the depth stop to increase the length of the discectomy channel having the second width; removing the tissue cutter from the discectomy channel; and, removing the disc tissue from the subject. It should be appreciated that selecting the desired depth is merely a translational setting on the cutting device and can be done before or after inserting the tissue cutter into the discectomy channel.

The controlled translation is a desired feature due to the critical nature of protecting the surrounding tissue that should not be cut. To obtain this, in some embodiments, the second cannula can be configured with a depth stop that can engage with at least one vertebral body adjacent to the discectomy. This engagement with the at least one vertebral body allows for the application of a controlled pressure that facilitates the controlled translation of the cutting element through the tissue. As such, the device can be configured with a second actuator for apply the controlled pressure to the depth stop through a second drive element. Although the use of a controlled pressure to the depth stop through an actuator is desirable, the pressure can be applied to the depth stop using any means, for example by merely manually translating the first cannula within the second cannula, for example, to translate the cutting element. Or, by actuating a second drive feature with an actuator to apply a controlled pressure through the actuator to translate the cutting element with respect to the depth stop. One of skill will appreciate that applying the controllable pressure to the depth stop will provide a controlled translational pressure that results in controllably translating the cutting head in the disc space to, in turn, controllably cut the disc tissue without cutting the critical adjacent tissues.

As such, in reference to the illustrations, namely FIGS. 4, 6, 20 and 21, the second control member 22 can be provided as the second actuator to drive threaded portion 62 as the second drive member of the first cannula. As effectively illustrated in these figures, the user may rotate the second control member 22, which causes the first cannula 14 and shaft 12 to translate in a proximal direction, effectively “pulling” the cutting flange 116 proximally through the disc space.

In some embodiments, as this proximal translation is occurring, the control pin 52 remains stationary but is effectively translating distally along the elongated recess 50 of the inner shaft 14 due to the proximal translation of the inner shaft 14 relative to the control pin 52. Proximal translation of the first cannula 14 and shaft 12 will cease when the distal end of the elongated aperture 74 abuts the control pin 52. At this point the cutting flange 116 has been proximally translated within the disc space to its fullest extent and the second control member 22 will turn no further (e.g. FIGS. 20-21). As the shaft 12 translates proximally relative to the control pin 52, the cantilever flange 48 is essentially “pulled” underneath the control pin 52 until the distal end of the flange 48 including the lip 49 is positioned under the control pin 52. During this action, the pin 52 deflects the distal end of the flange away from the elongated aperture 74, enabling further distal translation of the shaft 12 upon rotation of the first control member 20. This causes the cutting element 18 to pivot proximally once again until the cutting flange 116 is at 180° orientation within the housing element 24 (e.g. FIG. 22). The disc material that has been dislodged by the cutting element 116 may then be removed from the disc space. In some embodiments, a fusion implant may be inserted, for example, after removal of the tissue from the disc space, followed by the introduction of bone graft material for fusing the vertebrae together.

One of skill will appreciate that the cut tissue can be removed using any tools or means known to those of skill. For example, the skilled artisan may use a curette, a rongeur, forceps, vacuum, or a combination thereof.

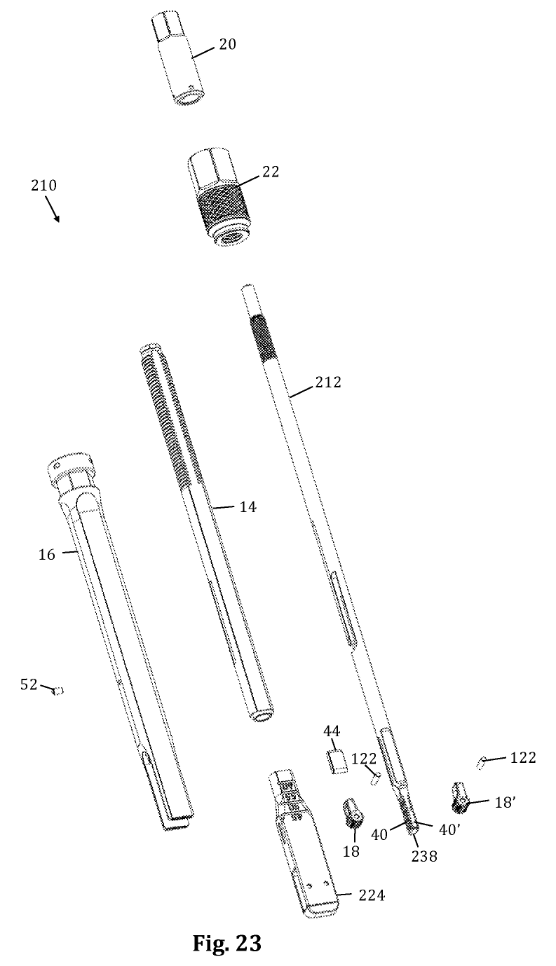

The sweeping cutters can have any number of cutting elements. For example, the cutters may have one blade on one side of the device, one blade on both sides of the device, more than one blade per side, or some combination thereof. The teachings of a unilateral tissue-cutting device, however, help illustrate the basic aspects and functionalities of the sweeping cutters. FIGS. 23-36 illustrate several examples of modification and/or features that may be made or added to enhance the functionality of the sweeping tissue cutter devices.

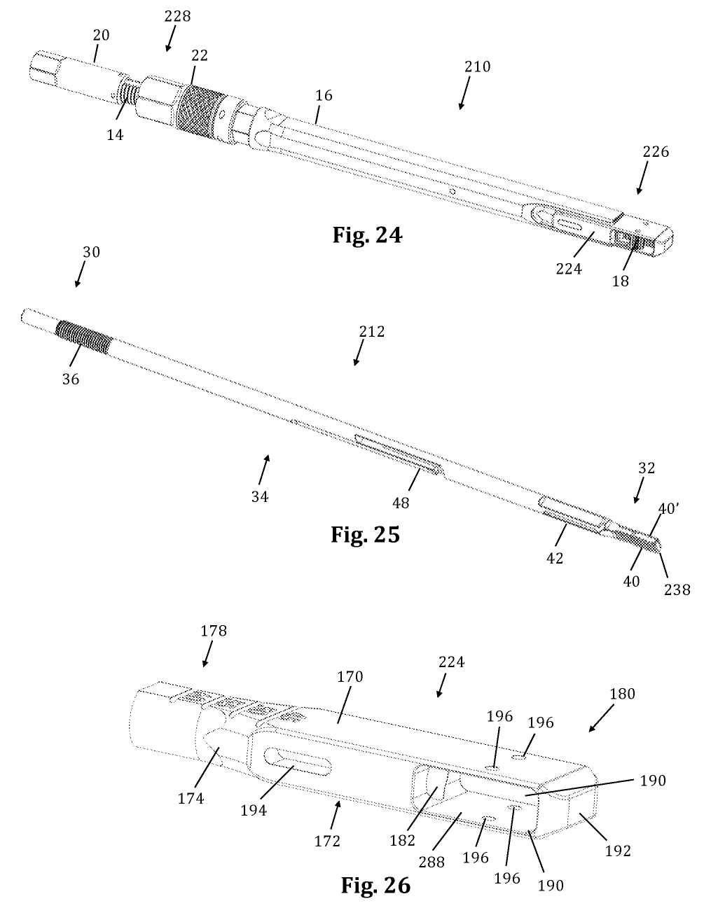

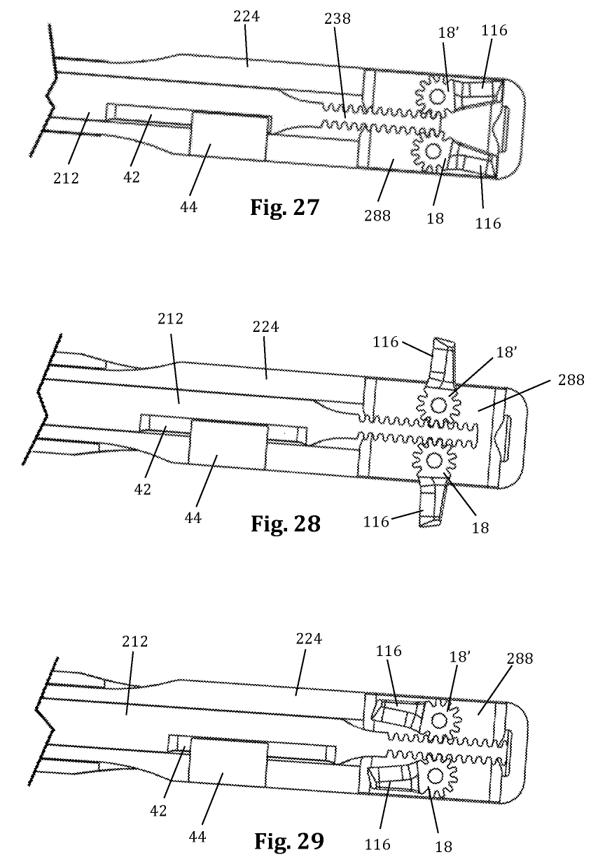

In some embodiments, the sweeping tissue cutter may be configured as a bilateral tissue cutter. FIGS. 23-29 illustrate an example of a bilateral sweeping tissue cutter 210, according to some embodiments, in which the surgical cutting device 210 is a bilateral tissue cutter configured to cut tissue on both sides of the instrument. FIG. 23 is an exploded view of another example of a bilateral sweeping tissue cutter, according to some embodiments. FIG. 24 is a perspective view of the bilateral sweeping tissue cutter of FIG. 23, according to some embodiments. FIG. 25 is a perspective view of an inner shaft member forming part of the bilateral sweeping tissue cutter of FIG. 23, according to some embodiments. FIG. 26 is a perspective view of a housing member forming part of the bilateral sweeping tissue cutter of FIG. 23, according to some embodiments. FIGS. 27-29 are top cross-sectional views of the bilateral sweeping tissue cutter of FIG. 23 at various stages of use, according to some embodiments.

Many of the components and features of the bilateral sweeping tissue cutter 210 are the same or similar in form and function to the corresponding components and features of the sweeping tissue cutter 10. Thus, the disclosure moving forward will focus on the components and features that are different, and the components and features that are the same will have the same numbering.

By way of example only, the bilateral sweeping tissue cutter 210 includes a shaft 212 positioned within a first cannula 14, which is itself positioned within a second cannula 16. The bilateral sweeping tissue cutter 210 further includes a pair of cutting elements 18, 18’ positioned near and interacting with the distal end of the shaft 212, and first and second control members 20, 22 positioned near the proximal end of the shaft 212. The first cannula 14 further includes a housing element 224 configured to facilitate insertion of the distal end of the device 210 into an intervertebral disc space while simultaneously protecting the distal end of the shaft 212 including the cutting elements 18,18’ from contact with patient tissue during insertion. The bilateral sweeping tissue cutter 210 has a distal end 226 comprising the distal end of the shaft 212, the cutting elements 18,18’, and the housing member 224. The bilateral sweeping tissue cutter 210 has a proximal end 228 comprising the respective proximal ends of the shaft 212, first cannula 14, and second cannula 16, as well as the first and second control elements 20,22. The bilateral sweeping tissue cutter 210 functions in the same manner as the sweeping tissue cutter 10 described above, namely actuating the first control member 20 causes the cutting elements 18,18’ to rotate from a first position in which the cutting elements 18,18’ are contained within the housing element 224 to a second position in which the cutting elements 18,18’ are protruding from the housing element 224. Once the cutting elements 18,18’ are in the second position, the second control member 22 may be actuated to maneuver the distal end 226 of the surgical cutting device 210 (e.g., including the shaft 212, cutting elements 18,18’, and housing member 224) axially in a proximal direction to drag the exposed cutting elements 18,18’ through the intervertebral disc space, thereby cutting or shaving the disc on both side of the shaft 12.

By way of example only, the shaft 212 comprises an axially aligned (e.g., along longitudinal axis L1), elongated, generally cylindrical member having a proximal end 30, distal end 32, and a central portion 34 extending between the proximal and distal ends. The proximal end 30 includes a threaded portion 36 sized and configured to engage the threaded portion of the first control member 20 as described above. The distal end 32 includes a distal flange 238 including a pair of gear racks 40,40’ located on opposite outward-facing sides of the distal flange 238. The gear racks 40.40’ each comprise a plurality of ridges or elongated gear teeth extending transverse to the longitudinal axis L1. The gear racks 40,40’ are configured to engage the gear teeth 118 of the cutting elements 18,18’ such that axial translation of the shaft 212 (e.g., in a proximal and/or distal direction) causes rotational pivoting of the cutting elements 18,18’. The distal end 32 of the shaft further comprises an axial recess 42 formed within the shaft, the axial recess 42 configured to receive a portion of a guide member 44 therein as described above. The central portion 34 includes an axially-aligned cantilever flange 48 with identical form and function as described above.

By way of example only, the housing element 224 comprises opposing first and second sides 170,172, each comprising a generally planar surface, opposing third and fourth sides 174,176, a proximal portion 178, and a distal portion 180. The proximal portion 178 includes an inner lumen 182 extending axially therethrough and configured to be continuous with the inner lumen 60 of the first cannula 14. The proximal portion 178 is seamlessly connected to inner cannula 14 as described above. The distal portion 180 comprises a chamber 288 wherein the first and second sides 170,172 form two sides of the chamber 288. The chamber 288 has a pair of opposing large apertures 190, 190’ formed in the third and fourth sides 174,176, respectively, providing access into the chamber 288 from the outside. The chamber 288 is sized and configured to contain and protect the distal end 32 of the shaft 212 and the cutting elements 18,18’ when the cutting elements 18,18’ are in an initial undeployed position (e.g. 0°, see FIG. 27) as well as when the cutting elements 18,18’ are in a final position (e.g. 180°, see FIG. 29). The distal portion 180 further includes a tapered leading end 192 to facilitate advancement of the bilateral sweeping tissue cutter 210 through patient tissue. The third side 174 includes a guide aperture 194 sized and shaped to receive the guide member 44 therein. The first and second sides 170,172 each include a pair of pivot apertures 196,196’ configured to secure one end of the pivot pin 122,122’ therein to secure the geared body 114 of the cutting elements 18,18’ within the chamber 288.

FIGS. 27-29 illustrate the positioning of several components of the bilateral sweeping tissue cutter 210 during various stages of a tissue cutting procedure (e.g. discectomy), according to some embodiments. As previously mentioned, operation of the bilateral sweeping tissue cutter 210 is very much the same as the operation of the sweeping tissue cutter 10 described above. By way of example, FIG. 27 illustrates the bilateral sweeping tissue cutter 210 in an initial state in which the cutting elements 18,18’ are in a first, initial position within the chamber 288 of the housing 224. FIG. 28 illustrates the bilateral sweeping tissue cutter 210 in which the cutting elements 18,18’ are in a second, deployed position in which the respective cutting flanges 116,116’ are at 90° relative to the shaft 212. FIG. 29 illustrates the bilateral sweeping tissue cutter 210 in which the cutting elements 18,18’ are in a third position in which the respective cutting flanges 116,116’ are at 180° within the chamber 288. Of particular importance in the instant example is that the cutting flanges 116,116’ are deployed simultaneously in a mirrored fashion on either side of the shaft 12.

In some embodiments, the housing members 24,224 may be modified to include an anti-migration feature to help prevent the housing member 24,224 from shifting within the disc space prior to cutting.

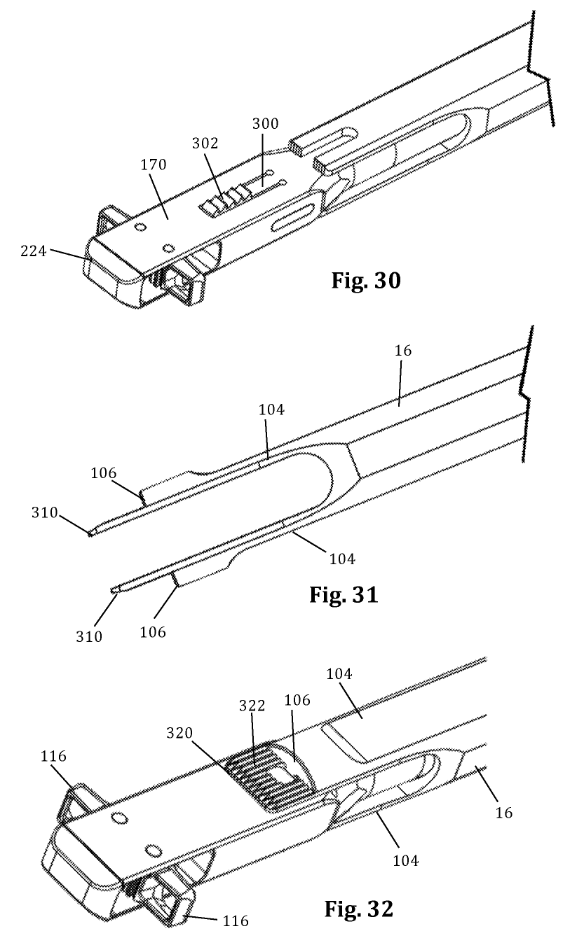

FIG. 30 is a perspective view of the bilateral sweeping tissue cutter of FIG. 23 in conjunction with an alternative example of a housing member, according to some embodiments. FIG. 30 illustrates an example of a bilateral sweeping tissue cutter 210 in which the housing member 224 includes a deflectable cantilever flange 300 with vertebral-contacting traction teeth 302 positioned within the first side 170. In such embodiments, deployment and/or retraction may be controlled in whole or in part by strategically placed recessed on the shaft 212. In some embodiments, an identical flange 300 may be positioned on the second side 172.

FIG. 31 is a perspective view of the distal end of another embodiment of a second cannula forming part of the sweeping tissue cutters taught herein, according to some embodiments. In some embodiments, as illustrated by way of example in FIG. 31, the second cannula 16 may include shims 310 that extend distally from the distal extensions 104 beyond the vertebral contact surfaces 106 and into the disc space, but adjacent the vertebral endplates. The shims 310 may provide for additional stability of the instrument during use by providing a non-invasive registration of outer cannula 16 to the vertebral bodies adjacent the target disc space.

FIG. 32 is a perspective view of the bilateral sweeping tissue cutter of FIG. 23 in conjunction with an alternative example of a second cannula, according to some embodiments. In some embodiments, as illustrated by way of example in FIG. 32, the second cannula 16 may include shims 320 that extend into the disc space, and have a plurality of longitudinally-oriented raised grooves 322 disposed on the outer-facing surfaces of the shims 320. In this example embodiment, the raised grooves 322 not only provide purchase and/or registration to the vertebral bodies, but also provide a height element in a situation in which the vertebral bodies are farther apart than normal to prevent the second cannula 16, and more importantly the cutting flanges 116,116’ from pivoting about the longitudinal axis L1 during use, as might be the case if the second cannula 16 did not securely engage the vertebral bodies.

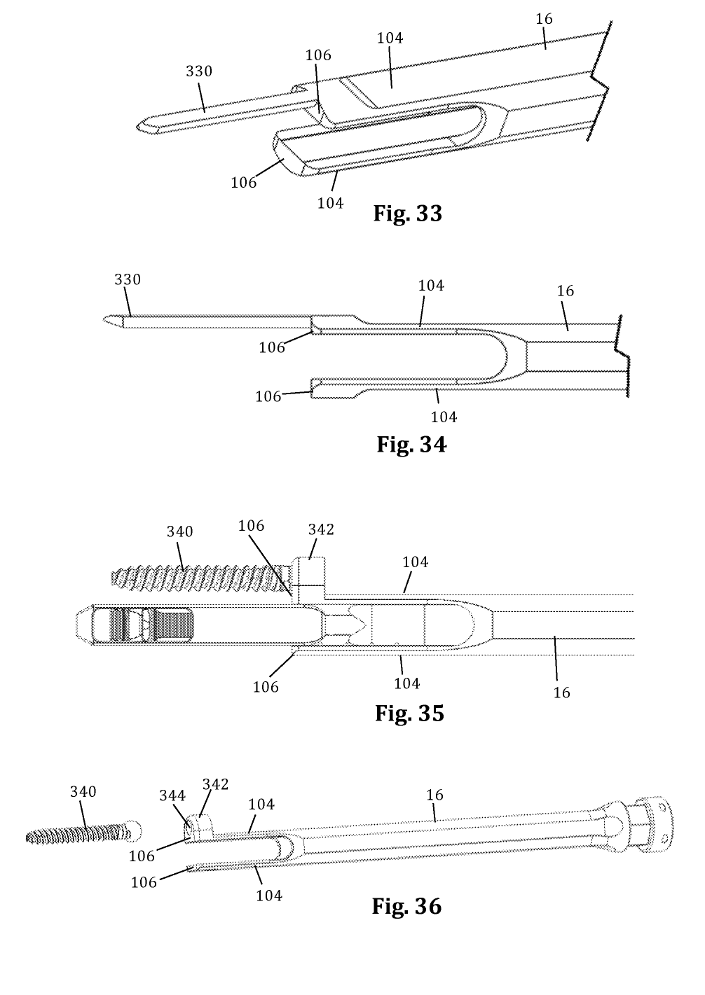

FIG. 33 is a perspective view of the distal end of another example of a second cannula forming part of the sweeping tissue cutters taught herein, according to some embodiments. FIG. 34 is a side plan view of FIG. 33, according to some embodiments. In some embodiments, as illustrated by way of example in FIGS. 33-34, the second cannula 16 may include an invasive anti-migration feature in the form of a barb, spike, or pin 330 extending distally from the vertebral contact surface 106 of one of the distal extensions 104. The barb 330 provides even more stability as it is driven into the cortical ring of one of the adjacent vertebral bodies.

FIG. 35 is a side plan view of an alternative example of a second cannula forming part of the sweeping tissue cutters taught herein, according to some embodiments. FIG. 36 is an exploded perspective view of the second cannula of FIG. 35, according to some embodiments. In some embodiments, as illustrated by way of example in FIGS. 35-36, the second cannula 16 may include an anti-migration feature in the form of a bone screw 340. This feature may be useful in situation where even more secure positioning is needed to ensure the sweeping tissue cutter 10/210 doesn’t move during use. By way of example only, the bone screw 340 may be associated with the second cannula 16 by passing through a screw aperture 344 on a retaining element 342 attached to or integrally formed with one of the distal extensions 104 such that the bone screw 340 extends distally beyond the vertebral contact surfaces 106.

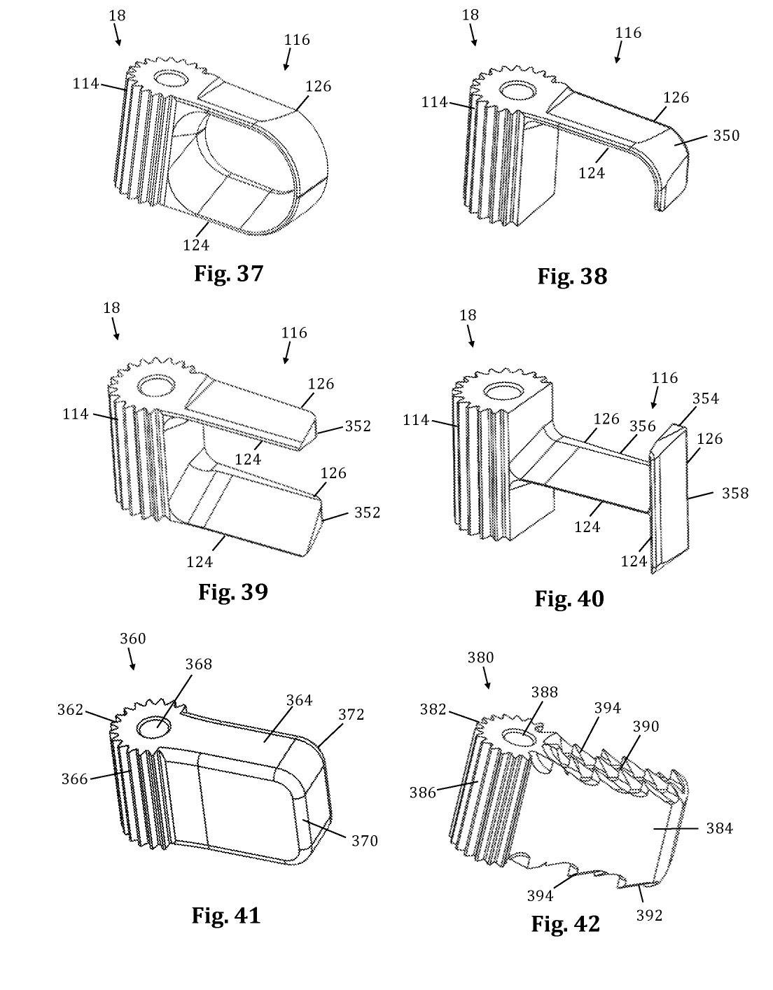

FIGS. 37-40 are perspective views of alternative examples of cutting elements forming part of the sweeping tissue cutters taught herein, according to some embodiments. In some embodiments, the cutting flange 18 may have different shape or configuration than as shown and described above. For example, FIG. 37 depicts an example of a cutting element 18 having a geared body 114 as described above and a generally oval-shaped cutting flange 116 comprising a sharp leading edge 124 configured for cutting through soft tissue and a generally planar or dull trailing edge 126 having a width that gives the cutting element a base for stability. FIG. 38 depicts an example of a cutting element 18 having a geared body 114 as described above and a cutting flange 116 comprising a curved member 350 extending laterally from the geared body 114. The curved member 350 comprises a sharp leading edge 124 configured for cutting through soft tissue and a generally planar or dull trailing edge 126 having a width that gives the cutting element a base for stability. FIG. 39 depicts an example of a cutting element 18 having a geared body 114 as described above and a cutting flange 116 comprising a pair of straight flanges 352 extending laterally from either end of the geared body 114. Each of the straight flanges includes a sharp leading edge 124 configured for cutting through soft tissue and a generally planar or dull trailing edge 126 having a width that gives the cutting element a base for stability. FIG. 40 depicts an example of a cutting element 18 having a geared body 114 as described above and a cutting flange 116 comprising a generally T-shaped member 354 extending laterally from the general middle of the geared body 114. The T-shaped member 354 includes a lateral or horizontal component 356 and a vertical or transverse component 358 positioned at the distal end of the horizontal component 356. By way of example only, the vertical component 358 may have a height dimension less than, greater than or equal to the height dimension of the geared body 114. Each of the lateral component 356 and the vertical component 358 comprises a sharp leading edge 124 configured for cutting through soft tissue and a generally planar or dull trailing edge 126 having a width that gives the cutting element a base for stability.

FIG. 41 is a perspective view of an example of a trial sizer configured for use with the sweeping tissue cutters taught herein, according to some embodiments. Trial elements can be used for sizing, and the trial elements can be used with any of the cutter device configurations taught herein For example, FIG. 41 illustrates an example of a trial element 360 configured for use with the sweeping tissue cutter 10, according to some embodiments. By way of example only, the trial element 360 may have a size dimension that is substantially similar to the size of the cutting element 18. The trial element 360 operates in a similar fashion to the cutting element 18 and in some embodiments may be interchangeable with, provided instead of, or provided in addition to the cutting element 18. After cutting with the cutting element 18, for example, the trial element 360 (e.g. on the same or different sweeping tissue cutter 10) may be used to ensure the disc space is sufficiently clear for the implant. By way of example, the trial element 360 of the current example embodiment includes a geared body 362 and a trial block 364 extending laterally from the geared body 362. The geared body 362 is generally cylindrical in shape and comprises a plurality of elongated gear teeth 366 configured to interact with the gear rack 40 of the shaft 12 such that axial translation of the shaft 12 (e.g. in a proximal and/or distal direction) causes rotational pivoting of the trial element 360, similar to the cutting element 18 described above. As such, in the present example the gear teeth 366 are oriented transverse to the longitudinal axis L 1 of the sweeping tissue cutter 10. The geared body 362 further comprises a central aperture 368 configured to receive the pivot pin 122 therethrough (e.g. FIG. 4). The pivot pin 122 is securely mated to the pivot apertures 196 of the housing element 24 (described above) and establishes an axis of rotation about which the trial element 360 pivots upon deployment. The trial block 364 of the current example embodiment comprises a generally rectangular member having a leading edge 370 and a trailing edge 372, each of which may be generally smooth and/or rounded to minimize tissue disruption. It should be noted that the trial block 364 may have any shape that corresponds to the relevant perimeter shape to be used in the procedure, including but not limited to rounded, squared, oval, etc.

FIG. 42 is a perspective view of an example of a rasp element configured for use with the sweeping tissue cutters taught herein, according to some embodiments. FIG. 42 illustrates an example of a rasp element 380 configured for use with the sweeping tissue cutter 10, according to some embodiments. By way of example only, the rasp element 380 may have a size dimension that is substantially similar to the size of the cutting element 18. The rasp element 380 operates in a similar fashion to the cutting element 18 and in some embodiments may be interchangeable with, provided instead of, or provided in addition to the cutting element 18. After cutting with the cutting element 18, for example, the rasp element 380 (e.g. on the same or different sweeping tissue cutter 10) may be used to score the vertebral endplates adjacent the disc space to create bleeding surfaces which will then encourage fusion with any bone graft (e.g. natural or synthetic) or other fusion-promoting material that may be used in the procedure. By way of example, the rasp element 380 of the current example embodiment includes a geared body 382 and a rasp flange 384 extending laterally from the geared body 382. The geared body 382 is generally cylindrical in shape and comprises a plurality of elongated gear teeth 386 configured to interact with the gear rack 40 of the shaft 12 such that axial translation of the shaft 12 (e.g., in a proximal and/or distal direction) causes rotational pivoting of the rasp element 380, similar to the cutting element 18 described above. As such, in the present example the gear teeth 386 are oriented transverse to the longitudinal axis L 1 of the sweeping tissue cutter 10. The geared body 382 further comprises a central aperture 388 configured to receive the pivot pin 122 therethrough (e.g. FIG. 4). The pivot pin 122 is securely mated to the pivot apertures 196 of the housing element 24 (described above) and establishes an axis of rotation about which the rasp element 380 pivots upon deployment. The rasp flange 384 of the current example embodiment comprises a shaped member (e.g. generally rectangular, trapezoidal, curved, or any shape needed to effectively prepare the vertebral endplates) having an upper-facing cutting surface 390 and a lower-facing cutting surface 392, each of which include a plurality of teeth 394 or other suitable scraping elements configured to disrupt the vertebral endplates.

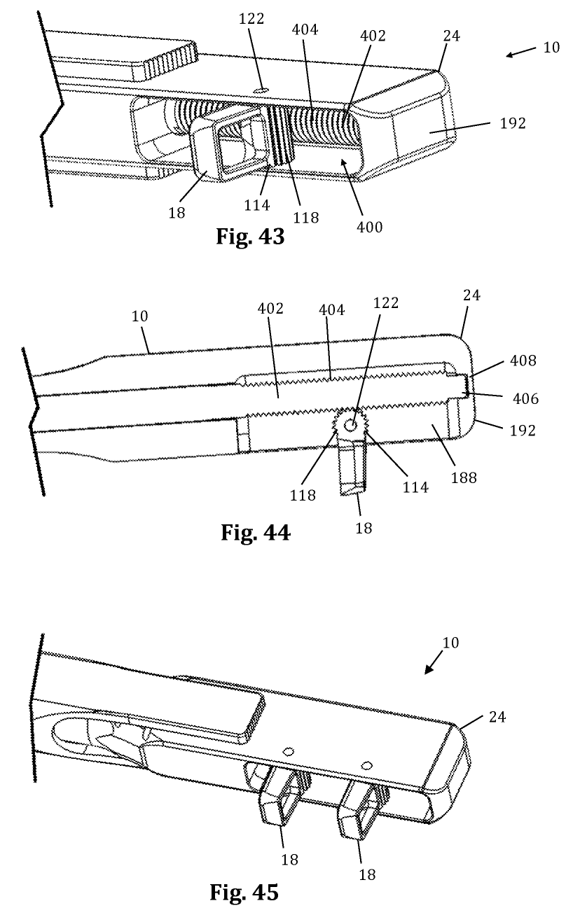

FIG. 43 is a perspective view of a sweeping tissue cutter having an alternative actuation mechanism, according to some embodiments. FIG. 44 is a top sectional view of FIG. 43, according to some embodiments. Each of the example embodiments of the sweeping tissue cutters 10, 210 shown and described above illustrate actuation of the cutting elements 18 as driven by a rack and pinion gear mechanism, by way of example only. It should be understood that the pivoting motion of the cutting elements 18 may be accomplished by any suitable mechanism, including but not limited to rack and pinion, worm gear, linkage arrangement, and the like. For example, FIGS. 43-44 illustrate an example of a sweeping tissue cutter 10 outfitted with a worm gear mechanism 400 for actuating the cutting element 18. By way of example, the worm gear mechanism comprises a drive shaft 402 configured for rotation without axial translation. In some embodiments, the drive shaft 402 may comprise the distal end 32 of the shaft 12. In some embodiments, the drive shaft 402 may be rotatably connected to the distal end 32 of the shaft 12. In any configuration, the drive shaft 402 includes a helical flange 404 extending around the circumference of the drive shaft 402 for substantially the length of the drive shaft 402. The helical flange 402 may be configured to interact with a complimentary feature (e.g. the gear teeth 118) on the geared body 114 of the cutting element 18 such that, upon rotation of the drive shaft 402, the cutting element 18 rotates about the pivot pin 122 as described above. The drive shaft 402 may further include a generally cylindrical distal post 406 configured to rotate within a distal recess 408 formed within the leading end 192 of the housing element 24, facing the chamber 188. The distal post 406 is rotatably secured within the distal recess 408 ensuring axial alignment of the shaft 12 during actuation of the drive shaft 402. Although shown and described by way of example only in relation to a unilateral, single cutter embodiment, the worm gear mechanism 400 (or any other actuation mechanism) may be used with any cutter configuration (e.g. unilateral, bilateral, single cutting element, multiple cutting elements, etc.) without departing from the scope of the disclosure.

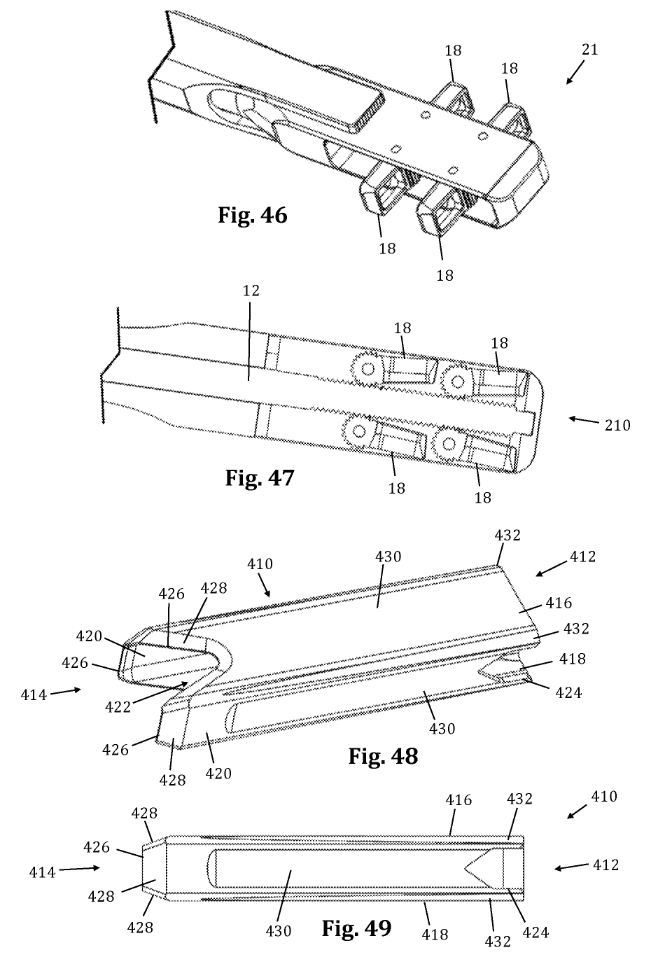

FIG. 45 is a perspective view of an example of the sweeping tissue cutters taught herein and configured with multiple cutting elements on a side of the cutter, according to some embodiments. FIG. 46 is a perspective view of an example of the sweeping tissue cutters taught herein and configured with multiple cutting elements on both sides of a bilateral cutter, according to some embodiments. FIG. 47 is a top sectional view of FIG. 46, according to some embodiments. FIGS. 45-47 illustrate several additional examples of cutting element configurations that the sweeping tissue cutter 10 (and/or sweeping tissue cutter 210) may employ. The sweeping tissue cutter 10 may be provided with any number and configuration of cutting elements 18 to suit the user’s needs. For example, FIG. 45 illustrates an example of a unilateral sweeping tissue cutter 10 having a pair of cutting elements 18 extending from the housing 24. FIGS. 46-47 illustrate an example of a bilateral surgical cutting device 210 having a pair of cutting elements 18 arranged symmetrically on either side of the shaft 12. Having multiple cutting elements 18 deployed at the same time removes the need to translate the shaft 12 proximally once the cutting element(s) has/have been deployed. In some embodiments, the number of cutting elements 18 per side of the device may be greater than two. In some embodiments, the arrangement of the cutting elements may be symmetrical. In some embodiments, the arrangement of cutting elements may be asymmetrical. In some embodiments, there may be a combination of cutting elements, trial elements, and/or rasp elements.

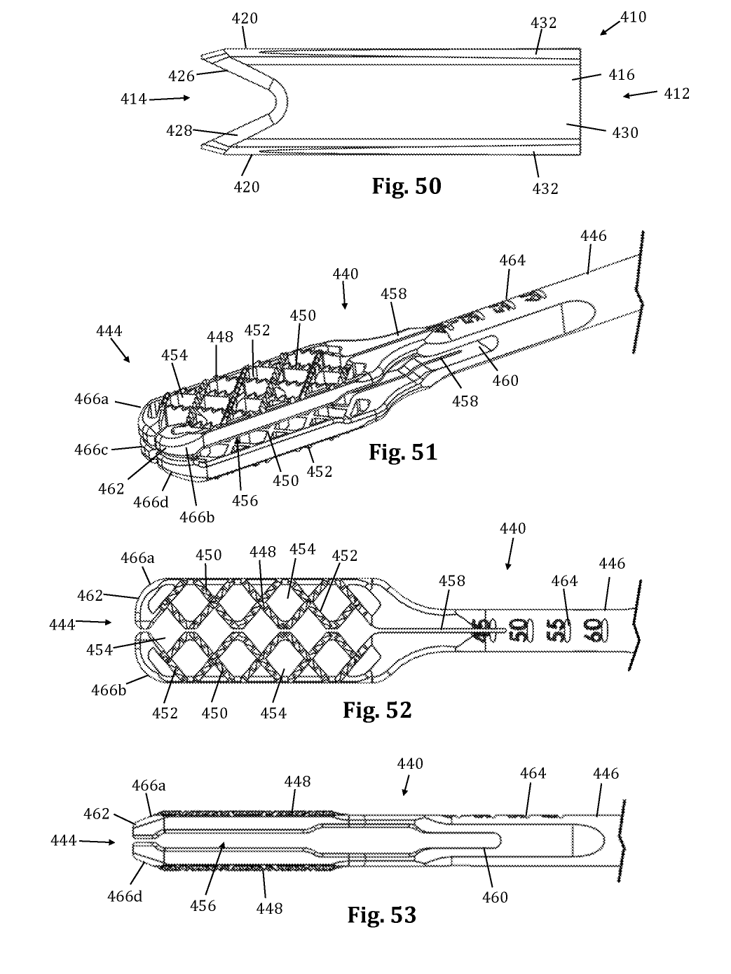

In some embodiments, a kit can be provided with a sweeping tissue cutter and channel cutter having a cross-sectional area sized to cut a channel out of a tissue for entry of the sweeping tissue cutter. FIG. 48 is a perspective view of a channel cutter configured for use with sweeping tissue cutters taught herein, according to some embodiments. FIG. 49 is a side plan view of the channel cutter of FIG. 48, according to some embodiments. FIG. 50 is a top plan view of the channel cutter of FIG. 50, according to some embodiments. FIGS. 48-50 illustrate an example of a channel cutter 410 configured for use with the sweeping tissue cutter 10, or the bilateral sweeping tissue cutter 210, according to some embodiments. By way of example, the channel cutter 410 is used to perform an initial channel discectomy, wherein a channel is created in the target disc space into which the sweeping tissue cutter 10/210 is inserted to enable the sweeping tissue cutter 10 to operate. As such, the channel created by the channel cutter 410 should have a size and shape complementary to the size and shape of the sweeping tissue cutter 10, but may also be larger or smaller than sweeping tissue cutter 10. By way of example only, the channel cutter 410 described herein has an elongated box-like shape, and may have length, width, and height dimensions that are similar to the length, width, and height dimensions of the housing element 24 of the sweeping tissue cutter 10 to be used in the procedure. Thus, a surgical kit may be provided with channel cutters 410 of various sizes to account for the size of the disc space and sweeping tissue cutter(s) 10 to be used.

By way of example, the channel cutter 410 has a generally rectangular cross-sectional shape, and includes a trailing or proximal end 412, a leading or distal end 414, a top wall 416, a bottom wall 418, a pair of opposing side walls 420, and a lumen 422 extending axially therethrough. The proximal end 412 includes an attachment element configured to attach the channel cutter 410 to a holder instrument (not shown). In the instant example, the attachment element comprises a pair of opposing recesses 424 formed within the side walls 420 at the proximal end, the recesses 424 configured to receive/engage a corresponding attachment element on a holder. Other attachment mechanisms are possible without departing from the scope of the disclosure. The distal end 414 includes a leading edge 426, comprising a sharp cutting edge for slicing through tissue (e.g. disc material).

In the instant example, the top and bottom walls 416, 418 comprise the long sides of the generally rectangular cross-sectional shape (and are not intended to attach a specific spatial orientation requirement during use, other than the channel cutter 410 being inserted into a disc space such that the top and bottom walls 416, 418 face the respective vertebral bodies adjacent the disc space), and together with the opposing sidewalls 420 define both the exterior perimeter shape of the channel cutter 410 and the interior perimeter of the lumen 422. The leading edge 426 comprises the distal end of each of the top wall 416, the bottom wall 418, and the sidewalls 420. By way of example, the leading edge may have a concave configuration along the top wall 416 and the bottom wall 418, and a generally straight configuration along the sidewalls 420. Each of the top wall 416, bottom wall 418, and sidewalls 420 further comprises a distal tapered surface 428 leading to the leading edge 426 and a recessed surface 430 extending proximally from the distal end. The tapered surface 428 helps to move cut disc material while the recessed surface 430 reduces friction between the channel cutter 410 and surrounding anatomy during use. Additional surface reliefs 432 may be formed in the corner edges (e.g. the junctions between the sidewalls 420 and the top and bottom walls 416, 418) to reduce friction between the channel cutter 410 and surrounding anatomy during use. The lumen 422 may be configured to contain and remove disc material that is cut during the initial channel discectomy procedure.

The system can also include a rasp cutter. In some embodiments, the rasp cutter can include a central chamber; a plurality of cutting elements around the central chamber; and, a plurality of open spaces between the plurality of cutting elements leading to the central chamber for collection and removal of tissue cut by the rasp. FIG. 51 is a perspective view of an example of a rasp instrument, according to some embodiments. FIG. 52 is a top plan view of the rasp instrument of FIG. 51, according to some embodiments. FIG. 53 is a side plan view of the rasp instrument of FIG. 52, according to some embodiments. FIGS. 51-53 illustrate an example of a rasp instrument 440. The rasp instrument 440 can be configured for cutting and removing tissue material (e.g. disc material, cartilage, etc.) during use so as to not impede continued cutting or rasping action. By way of example only, the rasp 440 includes a proximal end comprising a handle (not shown), a distal end 444, and an elongated shaft 446 extending between the proximal and distal ends. The distal end 444 includes a plurality of cutting surfaces 448 that are formed (by way of example only) by a plurality of cross-hatched fins 450 each featuring a cutting element 452 configured to cut and dislodge tissue material during use (e.g. including but not limited to teeth, serrations, roughened surfaces, abrasive surfaces, and/or sharpened edges). The cross-hatch configuration of the fins 450 creates a plurality of open spaces 454 leading into a central chamber 456 configured for collecting cut and dislodged tissue material for removal during use. The leading edge 462 of the distal end 444 may include one or more rounded or tapered surface to minimize trauma to surrounding tissue during use. The shaft 446 may have a plurality of depth markings 464 to inform the user how far across the disc space the distal end of the rasp is at any given time.

In some embodiments, the distal end 444 may be split in one or more configurations (e.g. horizontally, vertically) allowing resulting segments to flex relative to each other and thereby enabling improved conformance of the cutting surfaces 448 to the relevant bony anatomy (e.g. vertebral endplates). In the example shown and described herein, the rasp instrument 440 is provided with a vertical split 458 and a horizontal split 460, resulting in four distinct cutting segments 466 (e.g. cutting segments 466a, 466b, 466c, 466d). However, as mentioned the distal end 444 may be provided with any number of splits (including zero) to fit the needs of the user.

### Expansion cutters

In some embodiments, the tissue cutter is an expansion cutter. The expansion cutter can include an expandable cutting head having separable cutting elements operably attached to a wedge assembly having a proximal wedge and a distal wedge; and, an inner shaft operably attached to the distal wedge and translatable within an outer shaft operably attached to the proximal wedge. The wedge assembly can expand the separable cutting elements when the proximal wedge and distal wedge are translated relative to one another through a translation of the inner shaft with respect to the outer shaft.

The expansion and contraction of the cutting elements occurs results in the cutting of tissue. As such, a method of removing disc tissue with the expansion cutter can include creating an initial discectomy channel having a first width; inserting the tissue cutter into the discectomy channel; expanding the separable cutting elements of the expandable cutting head to cut the disc tissue and increase the discectomy channel to a second width; collapsing the separable cutting elements; removing the tissue cutter from the discectomy channel; and, removing the disc tissue from the subject.

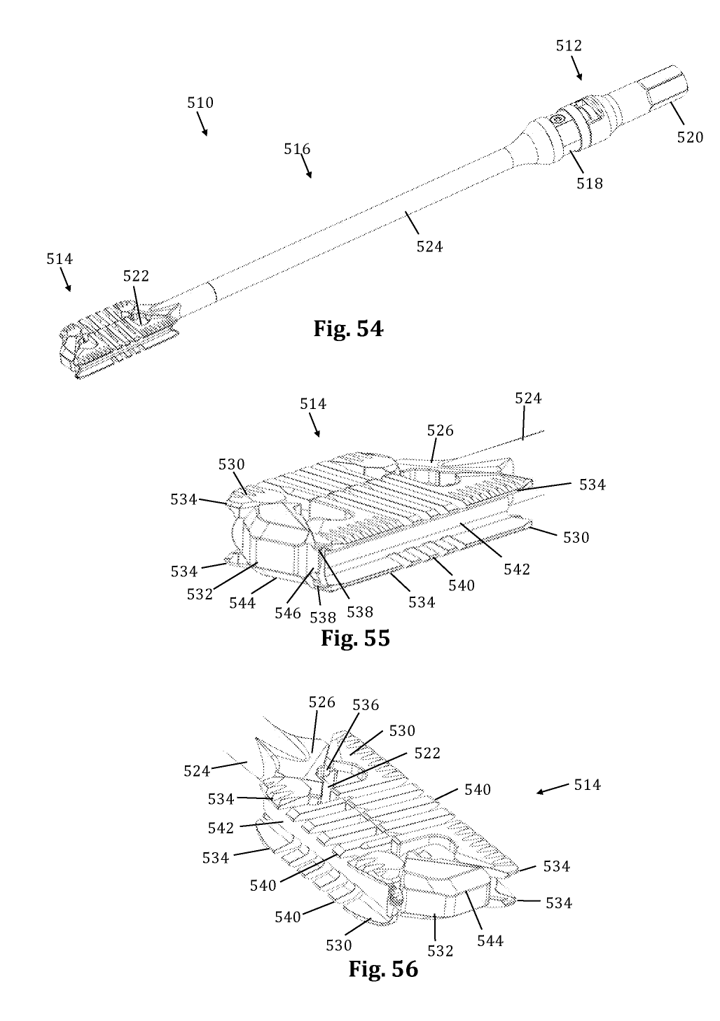

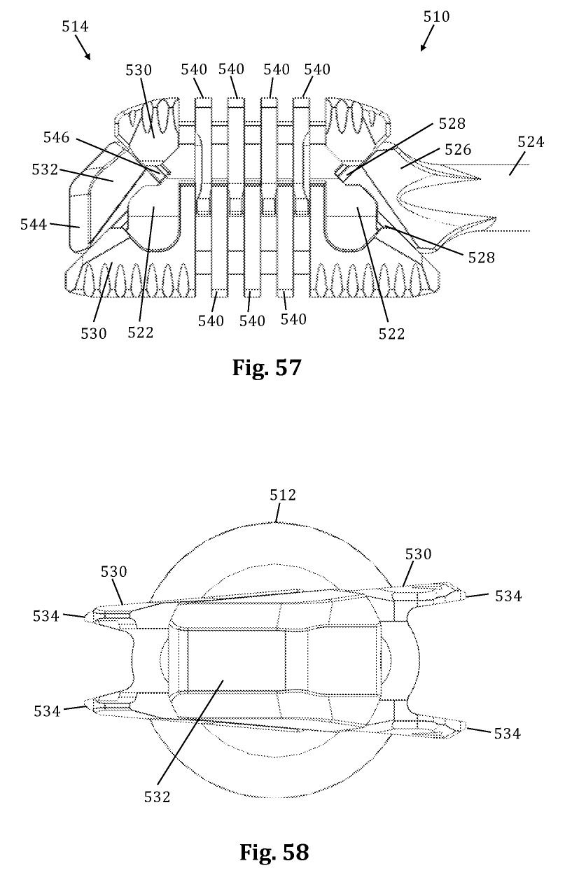

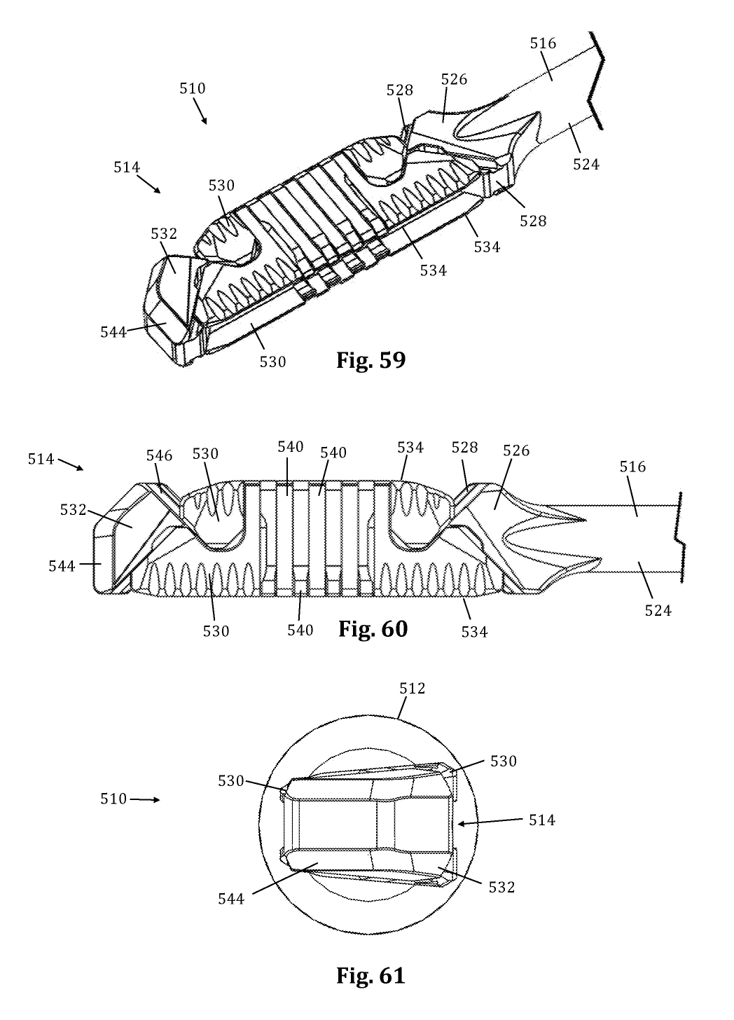

FIGS. 54-61 illustrate an example of expansion cutters, according to some embodiments. FIG. 54 is a perspective view of an expansion cutter, according to some embodiments. FIGS. 55-56 are perspective views of the distal end of the expansion cutter of FIG. 54, according to some embodiments. FIG. 57 is a top plan view of the distal end of the expansion cutter of FIG. 54, according to some embodiments. FIG. 58 is an end plan view of the distal end of the expansion cutter of FIG. 54, according to some embodiments. FIG. 59 is another perspective view of the distal end of the expansion cutter of FIG. 54, according to some embodiments. FIG. 60 is another top plan view of the distal end of the expansion cutter of FIG. 54, according to some embodiments. FIG. 61 is another end plan view of the distal end of the expansion cutter of FIG. 54.

As shown, the expansion cutters have an expandable cutting head that includes cutting elements operably attached to an expansion mechanism. The expandable cutting head can be configured to expand in any desired direction. In some embodiments, the expansion can be in the lateral direction, in some embodiments, the expansion can be in the vertical direction. In some embodiments, the expansion cutters can be configured to expand laterally, vertically, longitudinally, or a combination thereof. The vertical direction can be cephalocaudal, in some embodiments, with respect to the anatomical planes of the human body. Likewise, the lateral direction can be transverse, in some embodiments, with respect to the anatomical planes of the human body. In some embodiments, the expansion cutter can be used in an intervertebral disc space. For example, the expansion cutter 510 can be configured for insertion into a channel cut in to the disc using channel cutter 410, for example, in an initial, contracted configuration. This is illustrated, for example, in FIGS. 59-61. A lateral expansion of the device can be used to dislodge disc material within the target site of the intervertebral disc space.

By way of example only, the expansion cutter 510 can include a proximal end 512, a distal end 514, and an elongated shaft 516 extending between the proximal and distal ends. The proximal end 514 can include a handle or handle attachment element 518 configured to enable a user to manually manipulate the instrument, and an actuator knob 520.

The elongated shaft 516 comprises an inner shaft 522 having a longitudinal axis extending through an outer shaft 524. The inner shaft 522 can be rigidly connected to the distal wedge 532, in some embodiments, and rotatably connected to the actuator knob 520, in some embodiments. The inner shaft 522 is configured to translate proximally within the outer shaft 524 upon actuation (e.g. rotation) of the actuator knob. The outer shaft 524 has a distal end 526 comprising a coupling element 528 configured to slidably engage complementary coupling elements 536 on the cutting elements 530 of the distal end 514. By way of example only, the coupling elements 528, 536 may comprise complementary mating surfaces, including complementary grooves, channels, recesses, tongue-and-groove connectors, and the like. It should be appreciated that the complementary mating surfaces can be slidably translatable.

In some embodiments, the distal end 514 comprises a plurality of cutting elements 530 slidably associated with the distal end 526 of the outer shaft and a distal wedge 532. By way of example only, the expansion cutter 510 is shown and described as having a pair of opposing cutting elements 530. However, one of skill will appreciate that the device can be configured with any desired number of cutting elements. In some embodiments, there are four cutting elements. In some embodiments, the expansion cutters can be configured to have 2, 3, 4, 5, 6, 7, 8, 9, 10, 11, 12, 13, 14, 15, 16, 17, 18, 19, 20 cutting elements, or more, the cutting elements being on one or both sides of the device. In some embodiments, the number of cutting elements on one side of the device can equal the number of cutting elements on the other side of the device; or, in some embodiments, the number of cutting elements on one side of the device may not equal the number of cutting elements on the other side of the device. In some embodiments, there may be cutting elements on only one side of the device. In some embodiments, each cutting element 530 can have a pair of cutting edges 534, which can be lateral cutting edges, a proximal coupling element 536, a distal coupling element 538, and a plurality of stabilizer elements 540. In some embodiments, the lateral cutting edges 534 can have sharp edges configured for cutting through soft tissue (e.g. intervertebral disc material in the current example). By way of example only, the lateral cutting edges 534 can extend laterally from upper-facing and lower-facing surfaces of the cutting elements 530, creating a tissue channel 542 between the cutting edges 534 configured to contain cut and dislodged tissue material during use. The distal coupling elements 538 are configured to slidably engage complementary coupling elements 546 on the distal wedge 532. By way of example, the stabilizer elements 540 may comprise a plurality of interdigitated flanges extending transverse to the longitudinal axis of the expansion cutter 510, however other configurations are possible.

The distal wedge 532 may have a tapered and/or rounded leading end 544 and a coupling element 546. The leading end 544 can be tapered and/or rounded to minimize trauma to surrounding tissue during insertion. The coupling element 546 can be configured for slideable coupling with the distal coupling element 538 of the cutting elements 530.

A user of the device can advance the expansion cutter 510 in a contracted position (e.g. FIGS. 59-61) along an operative corridor and into an initial discectomy channel formed within an intervertebal disc (by way of example). The user can then translate the distal wedge with respect to the proximal wedge to expand the cutting head. In some embodiments, the user can actuate an actuator knob 520, which causes the inner shaft 522 to translate proximally relative to the outer shaft 524, thereby pulling the distal wedge 532 in a proximal direction. As the distal wedge 532 is pulled in a proximal direction, the cutting elements 530 are forced apart in a transverse direction, which pushes the cutting edges 534 into surrounding tissue (e.g., disc material), thereby cutting the desired amount of tissue for removal from the target site.

### Pull cutters

In some embodiments, the tissue cutter is a pull cutter. The pull cutter can include a spacer having an elongated body and a depth stop; and, a cutter translatable with respect to the spacer, the cutter having an elongated body and a proximal facing cutting element extending away from the depth stop, the proximal facing cutting element configured to cut tissue when pulling the cutter in a proximal direction. The pull cutter can further comprise an actuator, in some embodiments, the actuator configured for pulling the cutter in a proximal direction for the cutting of the tissue. The pulling can include translating the cutter with respect to the spacer and applying pressure against the depth stop for a controllable pulling of the cutter in the proximal direction.

The controlled pulling of the cutter results in the cutting of the tissue for removal. As such, a method of removing disc tissue with the pull cutter can include creating an initial discectomy channel having a first width; inserting the tissue cutter into the discectomy channel; translating the spacer in a distal direction with respect to the cutter and into the opening, the translating including applying pressure to the depth stop for a controllable pulling of the cutter in the proximal direction to cut the disc tissue and increase the discectomy channel to a second width; removing the tissue cutter from the discectomy channel; and, removing the disc tissue from the subject.

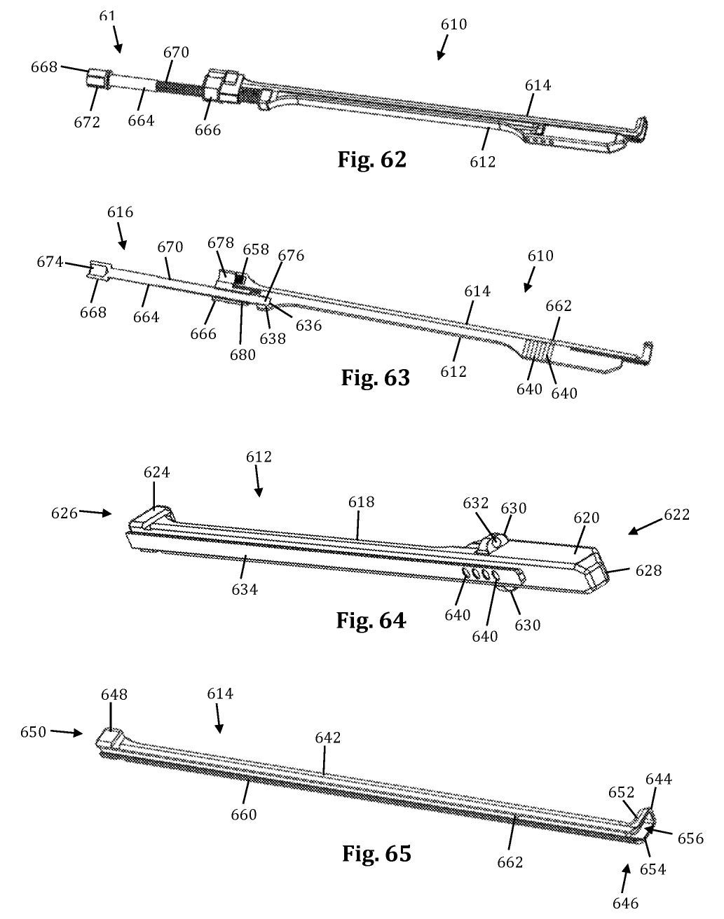

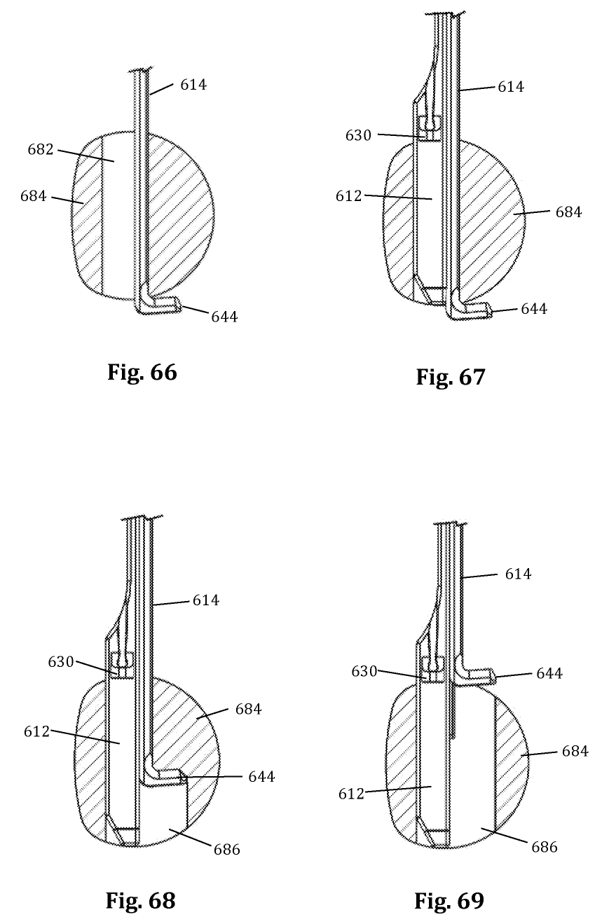

FIGS. 62-69 illustrate an example of a pull cutter 610, according to some embodiments. FIG. 62 is a perspective view of a pull cutter, according to some embodiments. FIG. 63 is a cross-sectional view of the pull cutter of FIG. 62, according to some embodiments. FIG. 64 is a perspective view of a spacer forming part of the pull cutter of FIG. 62, according to some embodiments. FIG. 65 is a perspective view of a cutter forming part of the pull cutter of FIG. 62, according to some embodiments. FIGS. 66-69 are cross-sectional top plan views of the pull cutter of FIG. 62 at various stages of use, according to some embodiments.

In some embodiments, the pull cutter 610 may include a spacer 612, a cutter 614, and an actuator 616. As described below, the cutter 614 can be slidably associated with the spacer 612, in some embodiments, and can be controlled by the actuator 616, in some embodiments. By way of example only, the pull cutter 610 can be a unilateral pull cutter configured for insertion into an annulotomy opening or an initial channel discectomy formed, for example, using channel cutter 410. The pull cutter can then be actuated to pull a cutting element proximally through the disc material.

In some embodiments, the spacer 612 can have an elongated shaft 618 with a spacer block 620 at a distal end 622 and an engagement feature 624 at a proximal end 626 for engaging with the actuator 616. The elongated shaft 618 may be sized and configured to extend axially through an operative corridor so that the distal end 622 extends into a surgical target site (e.g., intervertebral disc space) and the proximal end 626 extends out of the entry wound.

The spacer block 620 may be sized and configured to pass through the operative corridor and into an intervertebral disc by way of an annulotomy opening and/or an initial channel discectomy. In some embodiments, the spacer block 620 may have a cross-sectional shape that corresponds to a cross-sectional shape of the intended implant. By way of example only, the spacer block 620 may have a cross-sectional shape that is rectangular, square, ovoid, circular, and/or polygonal. In some embodiments, the spacer block 620 may have a tapered leading end 628 to facilitate smooth advancement along the operative corridor and into the target intervertebral disc. In some embodiments, the spacer block 620 may include one or more depth stop features 630 configured to impede advancement of the spacer block 620 beyond a predetermined point. In some embodiments, the depth stop feature 630 comprises one or more flanges 630 extending from the spacer block 620 sized and configured to abut the vertebral bodies adjacent the target disc space. In some embodiments, the spacer block 620 may include a depth stop feature 630 on a superior face (e.g. to engage a superior vertebral body), an inferior face (e.g. to engage an inferior vertebral body), or both superior and inferior faces. In some embodiments, the depth stop feature 630 may include one or more apertures 632 configured to receive an anchor element (e.g. screw, pin, nail, staple, etc.) to secure the spacer block 620 to the vertebral body.

In some embodiments, the spacer 612 may include an elongated rail 634 to facilitate slideable engagement with the cutter 614. By way of example only, the elongated rail 634 of the instant example comprises a dovetailed or tongue-and-groove association with the elongated track 660 of the cutter 614. The elongated rail 634 can be positioned on a medial side (e.g., the side that is facing or engaging with the cutter 614) of the spacer 612 and may extend substantially the length of the spacer 612, for example, from the proximal end 626 to the distal end 622. In some embodiments, the elongated rail 634 can extend from the proximal end 626. In some embodiments, the elongated rail 634 can extend distally at least partially along the spacer block 620, but not fully to the distal end 622.

In some embodiments, the engagement feature 624 may comprise a mating surface 636 that may optionally be formed within a proximal recess 638. By way of example only, the mating surface 636 may be generally smooth and configured to engage the distal end 676 of the actuator shaft 670 such that the actuator shaft 670 rotates while in contact with the mating surface 636 without causing movement of the spacer 612. In some embodiments, the proximal recess 638 may be sized and configured to receive at least a portion of the actuator shaft 670 therein to capture the distal end 676 of the actuator shaft 670 and prevent displacement of the actuator shaft 670 during use. By way of example, one or both of the distal end 676 of the actuator shaft 670 and the proximal recess 638 may be unthreaded.

In some embodiments, the spacer 612 may include one or more transverse through-holes 640 formed therein near the distal end 622. By way of example only, the spacer 612 of the present example includes four transverse through-holes 640 extending through the elongated rail 634 and the spacer block 620, although any number of transverse through-holes 640 may be formed in the spacer 612 as desired. The transverse through-holes 640 may aligned with one or more transverse through-holes 662 on the cutter 614 to give the user a visual indication of the relative positioning of the spacer 612 and cutter 614 (e.g., using fluoroscopy), as well as to help indicate what size of implant is needed.

By way of example only, the cutter 614 comprises an elongated shaft 642 having a cutting element 644 at a distal end 646 and a coupling feature 648 at a proximal end 650 for engaging with the actuator 616. The elongated shaft 642 may be sized and configured to extend axially through an operative corridor so that the distal end 646 extends into a surgical target site (e.g., intervertebral disc space) and the proximal end 650 extends out of the entry wound.

The cutting element 644 can also be referred to as a cutting flange 644, which can extend generally perpendicular from the shaft 642, or transverse to the longitudinal axis of the shaft. In some embodiments, the cutting flange 644 may extend at an angle greater than or less than 90º relative to the shaft 642. The cutting flange 644 may have any shape suitable to create the desired discectomy, including but not limited to (and by way of example only) rectangular, square, circular, ovoid, and polygonal. In some embodiments, the cutting flange 644 comprises a frame having a leading edge 652, a trailing edge 654, and a central opening 656. The leading edge 652 comprises a sharp edge configured for cutting through soft tissue (e.g. intervertebral disc material in the current example). The trailing edge 654 comprises a generally planar or dull edge having a width that gives the cutting element a base for stability. The central opening 656 is substantially large (e.g. such that the leading edge 652 represents the perimeter of the opening 656) to allow the cutting element 644 to dislodge a substantial amount of tissue material as the cutting element 644 advances through the tissue without simultaneously removing the tissue from the space. In this embodiment, the leading edge 652 is proximal-facing because the surgical cutting device 610 is configured as a pull cutter, and the leading or cutting edge 652 is pulled proximally through the intervertebral disc.

In some embodiments, cutter 614 may be secured to the coupling element 666 of the actuator 616 by any suitable mechanism that facilitates assembly during use, including but not limited to a screw, pin, snap-fit, and the like. By way of example only, the coupling feature 648 at the proximal end 650 may comprise a threaded recess 658, for example, to threadedly engage with a mating screw (not shown), to securely couple the cutter 614 with the coupling element 666 of the actuator 616.

In some embodiments, the cutter 614 may include an elongated track 660 to facilitate slideable engagement with the spacer 612. By way of example only, the elongated track 660 of the instant example comprises a dovetailed or tongue-and-groove association with the elongated rail 634 of the cutter 614. The elongated track 660 is positioned on a medial side of the cutter 614. In some embodiments, the medial side is the side that is facing or engaging with the spacer 612. In some embodiments, the elongated track 660 may extend substantially the length of the cutter 614, for example, from the proximal end 650 to the distal end 646.

In some embodiments, the cutter 614 may include one or more transverse through-holes 662 formed therein near the distal end 646. By way of example only, the cutter 614 of the present example includes one transverse through-hole 662 extending through the elongated track 660, although any number of transverse through-holes 662 may be formed in the cutter 614 as desired. The transverse through-holes 662 may align with one or more transverse through-holes 640 on the spacer 612 to give the user a visual indication of the relative positioning of the spacer 612 and cutter 614 (e.g., using fluoroscopy), as well as to help indicate what size of implant is needed.

In some embodiments, the actuator 616 may comprise any mechanism suitable to exert a distal force on the spacer 612 while simultaneously exerting a proximal force on the cutter 614, including but not limited to an actuation screw, a squeeze handle actuator, and the like. In some embodiments, the actuator 616 may comprise an actuation screw 664 and a coupling element 666. By way of example only, the actuation screw 664 may include a head 668 and a shaft 670. In some embodiments, the head 668 may include shaped outer surface 672 configured to engage with a driver, a proximal recess 674 configure to engage with a driver, or both. In some embodiments, the shaft 670 is at least partially threaded and includes a distal end 676 having an abutment surface configured to engage the mating surface 636 of the spacer 612 such that the actuator shaft 670 rotates while in contact with the mating surface 636 without causing movement of the spacer 612, as described above.

In some embodiments, the coupling element 666 is configured to securely couple the actuator 616 to the cutter 614. By way of example only, the coupling element 616 may include a coupling aperture 678 in axial alignment with the threaded recess 658 of the cutter 614 and configured to enable passage of a mating screw (for example) through the coupling element 666 and into the cutter 614. The coupling element 616 may further comprise a threaded channel 680 configured to threadedly engage the at least threaded portion of the actuator shaft 670.

FIGS. 66-69 illustrate the pull cutter 610 in use, according to some embodiments. By way of example, after an operative corridor has been established to the surgical target site, an initial discectomy channel 682 may be formed within the target intervertebral disc 684, for example, and this can be done using the channel cutter 410 described above. The cutter 614 may then be inserted through the channel 682 so that the cutting element 644 is positioned beyond the distal opening of the channel 682. The cutter 614 may then be biased within the channel 682 toward the side of the disc to be cut, as shown by way of example only in FIG. 66. The spacer 612 may then be coupled to the cutter 614 (if not already coupled) by mating the elongated rail 634 with the elongated track 660. The spacer 612 may then be translated along the cutter 614 until the one or more depth stops 630 abut the vertebra, for example as shown in FIG. 67. At this point, the actuator 616 may be coupled to the cutter 614 by way of a mating screw or other coupling feature, as described above, and positioned such that the actuation screw 664, for example, is in contact with the mating surface 636 of the spacer 612. The user may then rotate the actuator 616, which causes the coupling element 666 of the actuator 616 to translate proximally along the actuator shaft 664. Since the coupling element 666 is secured to the cutter 614, the coupling element 666 in turn pulls the cutter 614 in a proximal direction, which in turn causes the cutting element 644 to translate proximately through the disc 684 to create an expanded discectomy channel 686, as shown by way of example in FIGS. 68-69. The user may then remove the surgical cutting device 610 from the operative corridor.

### Push cutters

In some embodiments, the tissue cutter is a push cutter. The push cutter can include a spacer having an elongated body and a depth stop; and, a cutter translatable with respect to the spacer, the cutter having an elongated body and a distal facing cutting element extending away from the spacer block, the cutting element configured for cutting tissue when pushing the cutter in a distal direction. One of skill will appreciate that “push” can mean any application of pressure in the distal direction, whether the pressure applied includes an impact, a cyclic application, or constant pressure. In some embodiments, the push cutter can receive a tapping pressure, sharp and repeated impulses of force, which pushes the cutter through the tissue in the distal direction, for example.

The pushing of the cutter results in the cutting of the tissue for removal. As such, a method of removing disc tissue with the push cutter can include creating an initial discectomy channel having a first width; inserting the tissue cutter into the discectomy channel; translating the spacer in a distal direction on the cutter and into the opening to the depth stop; pushing the cutter distally along the spacer to cut the disc tissue and increase the discectomy channel to a second width; removing the tissue cutter from the discectomy channel; and, removing the disc tissue from the subject.

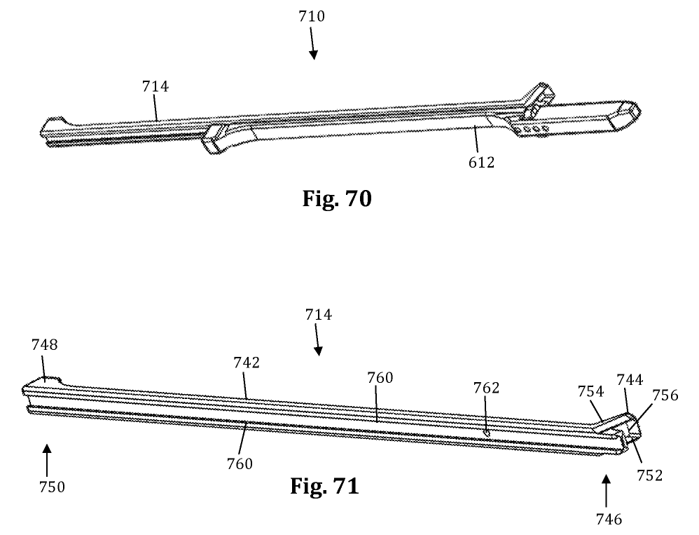

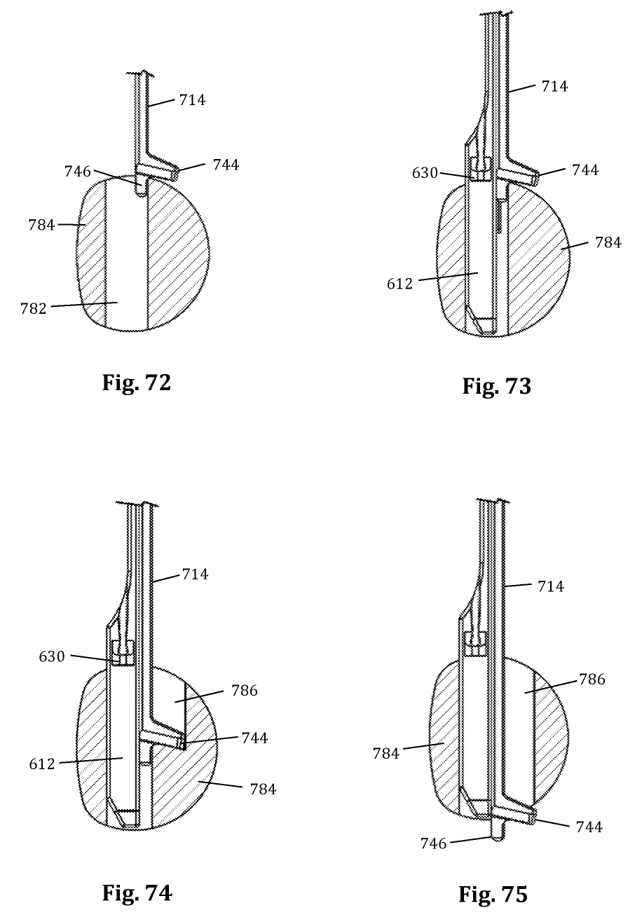

FIGS. 70-75 illustrate an example of a push cutter 710, according to some embodiments. FIG. 70 is a perspective view of an example of a push cutter, according to some embodiments. FIG. 71 is a perspective view of a cutter forming part of the push cutter of FIG. 70, according to some embodiments. FIGS. 72-75 are cross-sectional top plan views of the push cutter of FIG. 70 at various stages of use, according to some embodiments. In some embodiments, the push cutter 710 may include a spacer 612 and a cutter 714. In some embodiments, the spacer 612 of the push cutter 710 can be identical to the spacer 612 of the surgical cutting device 610 described above, and so the details won’t be repeated for this example. The cutter can be configured using any means that will translate the cutter with respect to the spacer. For example, the cutter 714 can be slideably associated with the spacer 612. In some embodiments, the surgical cutting device 710 can be a unilateral push cutter configured for partial insertion into an annulotomy opening or an initial channel discectomy (for example formed using channel cutter 410 described above) and then actuated to push a cutting element distally through the disc material.

By way of example only, the cutter 714 comprises an elongated shaft 742 having a cutting element 744 at a distal end 746 and an actuation feature 748 at a proximal end 750. The elongated shaft 742 may be sized and configured to extend axially through an operative corridor so that the distal end 746 extends into a surgical target site (e.g., intervertebral disc space) and the proximal end 750 extends out of the entry wound.

By way of example, only, the cutting element 744 can have a cutting flange 744 extending generally perpendicularly from the shaft 742, or transverse to the longitudinal axis of the shaft. In some embodiments, the cutting flange 744 may extend at an angle greater than or less than 90º relative to the shaft 742. The cutting flange 644 may have any shape suitable to create the desired discectomy, including but not limited to (and by way of example only) rectangular, square, circular, ovoid, and polygonal. In some embodiments, the cutting flange 744 comprises a frame having a leading edge 752, trailing edge 754, and a central opening 756. The leading edge 752 comprises a sharp edge configured for cutting through soft tissue (e.g. intervertebral disc material in the current example). The trailing edge 754 comprises a generally planar or dull edge having a width that gives the cutting element a base for stability. The central opening 756 is substantially large (e.g. such that the leading edge 752 represents the perimeter of the opening 756) to allow the cutting element 744 to dislodge a substantial amount of tissue material as the cutting element 744 advances through the tissue without simultaneously removing the tissue from the space. In this embodiment, the leading edge 752 is distal-facing because the push cutter 710 is configured as a push cutter, and the leading or cutting edge 752 is pushed distally through the intervertebral disc.

In some embodiments, the cutter 714 may include an elongated track 760 to facilitate slideable engagement with the spacer 612. By way of example only, the elongated track 760 of the instant example comprises a dovetailed or tongue and groove association with the elongated rail 634 of the cutter 714. The elongated track 760 is positioned on a medial side (e.g., the side that is facing or engaging with the spacer 712) of the cutter 714 and may extend substantially the length of the cutter 714, for example from the proximal end 750 to the distal end 746.

In some embodiments, the cutter 714 may include one or more transverse through-holes 762 formed therein near the distal end 746. By way of example only, the cutter 714 of the present example includes one transverse through-hole 762 extending through the elongated track 760, however any number of transverse through-holes 762 may be formed in the cutter 714 as desired. By way of example, the transverse through-holes 762 may align with one or more transverse through-holes 640 on the spacer 612 give the user a visual indication of the relative positioning of the spacer 612 and cutter 714 (e.g. using fluoroscopy), as well as to help indicate what size of implant is needed.

By way of example, the actuation feature 748 may comprise any mechanism suitable to exert a distal force on the cutter 714 to push the cutting element 744 through the intervertebral disc. In some embodiments, the actuation feature 748 may comprise a tamping surface that the user may tamp to exert a distal force on the cutter 714.

FIGS. 72-75 illustrate the push cutter 710 in use, according to some embodiments. By way of example, after an operative corridor has been established to the surgical target site, an initial discectomy channel 782 may be formed within the target intervertebral disc 784, for example, using the channel cutter 410 described above. The cutter 714 may then be inserted through the channel 782 so that the distal tip 746 is at least partially extending into the channel 782, and the cutting element 744 is positioned outside and proximal to the proximal opening of the channel 782. The cutter 714 may then be biased within the channel 782 toward the side of the disc to be cut, as shown in FIG. 72, for example. The spacer 612 may then be coupled to the cutter 714, if not already coupled, by mating the elongated rail 634 with the elongated track 760. The spacer 612 may then be slideably advanced along the cutter 714 until the one or more depth stops 630 abut the vertebra, as shown in FIG. 73, for example. The user may then tap the cutter 714, in some embodiments, which causes the cutter 714 translate distally along the spacer 612, which in turn pushes the cutting element 744 distally through the disc 784 to create an expanded discectomy channel 786, as shown in FIGS. 74-75. The user may then remove the surgical cutting device 710 from the operative corridor.

We claim:

1. A tissue cutter, comprising:
an elongated body having a longitudinal axis, and comprising at least one depth stop configured to abut at least one vertebral body adjacent the intervertebral disc;
a spacing element sized and configured to substantially fill an initial discectomy channel formed by the tissue cutter; and,
a cutting element comprising one or a plurality of cutting flanges, each cutting flange having at least one cutting edge for expanding the initial discectomy channel.

2. The tissue cutter according to claim 1, wherein the cutting element is positioned at least partially within an internal cavity of the spacing element.

3. The tissue cutter according to claim 2, wherein the cutting element comprises at least one cutting flange positioned along a lateral edge of the elongated body and transverse to the longitudinal axis, the at least one cutting flange comprising a distally oriented leading cutting edge and a proximally oriented trailing edge.

4. The tissue cutter according to claim 3, wherein:
the cutting element further comprises a geared body having a central axis;
the spacing element further comprises a rotational connection with the cutting element; and,
the tissue cutter further comprises a first cannula in operable connection with the internal cavity and a drive element positioned within the first cannula and in operable contact with the geared body of the cutting element, the drive element configured for pivoting the geared body in the internal cavity for a sweeping arc motion of the cutting flange when actuating the drive element.

5. The tissue cutter according to claim 4, further comprising a second cutting element positioned at least partially within the internal cavity, the second cutting element having a geared body in operable connection with the drive element and a cutting flange comprising a cutting edge.

6. The tissue cutter according to claim 5, wherein the cutting element and the second cutting element are positioned on a same side of the spacing element.

7. The tissue cutter according to claim 5, wherein the cutting element and the second cutting element are positioned on opposing sides of the spacing element.

8. The tissue cutter according to claim 5, wherein the drive element is configured to pivot the cutting element and the second cutting element simultaneously.

9. A tissue cutter comprising:
an elongated body having a longitudinal axis; and,
a cutting element comprising one of: (a) one or a plurality of cutting flanges, each cutting flange comprising at least one cutting edge, wherein the at least one cutting edge is selected from the group consisting of a distally oriented cutting edge that is transverse to the longitudinal axis, a proximally oriented cutting edge that is transverse to the longitudinal axis, and a combination thereof; or (b) a lateral cutting edge that is oriented parallel to the longitudinal axis.

10. The tissue cutter according to claim 9, the tissue cutter further comprising a spacer having an elongated body and a depth stop, and wherein the cutting element extends away from the depth stop and comprises a cutting flange comprising a distally oriented cutting edge that is transverse to the longitudinal axis, or a proximally oriented cutting edge that is transverse to the longitudinal axis, and wherein, the cutting edge is configured to cut tissue when the tissue cutter is translated along a cutting path from one of proximal to distal or distal to proximal along the elongated body such that the at least one cutting flange contacts with the intervertebral disc to cut the disc tissue along the cutting path.

11. The tissue cutter according to claim 9, wherein the tissue cutter further comprises:
an expandable cutting head having separable cutting elements operably attached to a wedge assembly having a proximal wedge and a distal wedge; and,
an inner shaft operably attached to the distal wedge and translatable within an outer shaft operably attached to the proximal wedge;
wherein, the wedge assembly laterally expands the separable cutting elements when the proximal wedge and distal wedge are translated relative to one another through a translation of the inner shaft with respect to the outer shaft, and wherein the cutting elements comprise a lateral cutting edge that is oriented parallel to the longitudinal axis.

12. A method of removing a disc tissue from an intervertebral disc of a subject, comprising the steps of:
creating an initial discectomy channel having a first width;
inserting a tissue cutter at least partially into the initial discectomy channel, the tissue cutter comprising:
an elongated body having a longitudinal axis and comprising at least one depth stop configured to abut at least one vertebral body adjacent the intervertebral disc;
a spacing element sized and configured to substantially fill the initial discectomy channel; and
a cutting element comprising one or a plurality of cutting flanges, each cutting flange comprising at least one cutting edge;
moving the cutting flange into contact with and through the intervertebral disc to cut contacted disc tissue;
removing the tissue cutter from the discectomy channel; and,
removing the remaining cut disc tissue from the subject to thereby expand the discectomy channel from the first width to a second width.

13. The method according to claim 12, wherein the cutting element is positioned at least partially within an internal cavity of the spacing element.

14. The method according to claim 13, wherein:
the cutting element is oriented transverse to the longitudinal axis and comprises a cutting flange comprising a leading cutting edge, and a trailing edge;
the cutting element further comprises a geared body having a central axis;
the spacing element further comprises a rotational connection with the cutting element; and,
the tissue cutter further comprises a first cannula in operable connection with the internal cavity and a drive element positioned within the first cannula and in operable contact with the geared body of the cutting element, the drive element configured for pivoting the geared body in the internal cavity for a sweeping arc motion of the cutting flange when actuating the drive element.

15. The method according to claim 14, wherein the step of moving the cutting flange into contact with and through the intervertebral disc to cut contacted disc tissue comprises actuating the drive element to pivot the geared body so that the cutting flange travels through the intervertebral disc in a sweeping arc motion.

16. The method according to claim 15, further comprising a second cutting element positioned at least partially within the internal cavity, the second cutting element having a geared body in operable connection with the drive element and a cutting flange comprising a cutting edge.

17. The method according to claim 16, wherein the cutting element and the second cutting element are positioned on a same side of the spacing element.

18. The method according to claim 16, wherein the cutting element and the second cutting element are positioned on opposing sides of the spacing element.

19. The method according to claim 16, wherein the drive element is configured to pivot the cutting element and the second cutting element simultaneously.

20. The method according to claim 14, wherein the step of moving the cutting flange into contact with and through the intervertebral disc to cut contacted disc tissue comprises moving the cutting flange in a sweeping arc motion through the intervertebral disc.
